(19) United States
(12) Patent Application Publication
(10) Pub. No.: US 2025/0163371 A1
(43) Pub. Date: May 22, 2025

(54) METHODS OF PRODUCING GLIAL CELLS AND USES THEREOF

(71) Applicant: StemCell Technologies Canada Inc., Vancouver (CA)

(72) Inventors: Abdulkadir Ozkan, Watertown, MA (US); Chun-I Wu, Watertown, MA (US); Devin Bridgen, Watertown, MA (US); Jonathan Gilbert, Watertown, MA (US)

(21) Appl. No.: 18/841,091

(22) PCT Filed: Feb. 22, 2023
(86) PCT No.: PCT/US2023/062998

§ 371 Date: Aug. 23, 2024

Related U.S. Application Data
(60) Provisional application No. 63/313,094, filed on Feb. 23, 2022.

(51) Int. Cl. C12N 5/079

(52) U.S. Cl. CPC C12N 5/0622; C12N 2506/03

(57) ABSTRACT

The present disclosure provides methods for reprogramming a cell into a glial cell (e.g., an oligodendrocyte progenitor cell), wherein the method comprises passing a cell suspension comprising the cell and a glial cell reprogramming factor through a constriction, wherein the constriction deforms the cell, thereby causing a perturbation of the cell such that the glial cell reprogramming factor enters the cell.

### CROSS-REFERENCE TO RELATED APPLICATION

This PCT application claims the priority benefit of U.S. Provisional Application No. 63/313,094, filed Feb. 23, 2022, which is herein incorporated by reference in its entirety.

### FIELD OF DISCLOSURE

The present disclosure relates generally to methods of producing glial cells (e.g., oligodendrocyte progenitor cells which can further differentiate into oligodendrocytes) through the use of one or more constrictions.

### BACKGROUND OF DISCLOSURE

Regenerative medicine based on cell replacement is on the cusp of major health impacts particularly for the treatment of demyelinating diseases. For instance, preclinical studies have suggested that transplanted neural progenitor cells could exert multifaceted therapeutic effects, including neuroprotective effects, via secretion of an array of factors with immunomodulatory and neurotrophic properties, as well as the ability to drive remyelination. However, significant challenges remain, particularly in manufacturing consistent, efficient, and cost-effective functional engraftable cells. While induced pluripotent stem cell (iPSC) differentiation and somatic cell transdifferentiation (i.e., direct reprogramming) are capable of producing various cells of interest, current methods are often cumbersome and inefficient. For instance, iPSC differentiation methods currently available can typically take up to several weeks and in some instances, even months to get to the desired terminal cell types. Furthermore, the resulting cell product is inevitably a heterogeneous population of cells with the desired cells present in wide range of frequency. Similarly, with somatic cell transdifferentiation, reprogramming by lentiviral vectors has been useful, but this method presents the risk of insertional mutagenesis. And, with methods such as electroporation and lipofection, they can negatively affect cell health and recovery of sensitive primary cells, as well as causing other cytotoxicity issues. Therefore, there remains a need for more efficient and cost-effective approaches to producing cells that can be used for various therapeutic applications, such as in regenerative medicine for the treatment of demyelinating diseases.

### SUMMARY OF DISCLOSURE

Provided herein is a method of producing a glial cell, comprising: passing a cell suspension, which comprises a population of cells, through a constriction under one or more parameters, wherein passing the cell suspension through the constriction under the one or more parameters deforms one or more cells of the population of cells, and thereby, causing a perturbation in the cell membrane of the one or more cells, and wherein the perturbation allows one or more glial cell reprogramming factors to enter the one or more cells, and thereby, reprograms the one or more cells into a glial cell. In some aspects, the glial cell comprises an oligodendrocyte progenitor cell (OPC), oligodendrocyte, or both.

In some aspects, contacting the population of cells with the glial cell reprogramming factor prior to the passing of the cell suspension through the constriction. In some aspects, contacting the population of cells with the glial cell reprogramming factor during the passing of the cell suspension through the constriction. In some aspects, the population of cells are contacted with the glial cell reprogramming factor during the passing of the cell suspension through the constriction. In some aspects, the above method comprises contacting the population of cells with the glial cell reprogramming factor after the passing of the cell suspension through the constriction. In some aspects, the population of cells are contacted with the glial cell reprogramming factor after the passing of the cell suspension through the constriction.

In some aspects, the glial cell reprogramming factor that can be used with the above methods comprises a nucleic acid, a polypeptide, a lipid, a carbohydrate, a small molecule, a metal-containing compound, an antibody, a transcription factor, a nanoparticle, a liposome, a fluorescently tagged molecule, or combinations thereof.

In some aspects, the nucleic acid comprises a DNA, RNA, or both. In some aspects, the DNA comprises a recombinant DNA, a cDNA, a genomic DNA, or combinations thereof. In some aspects, the RNA comprises a siRNA, a mRNA, a microRNA (miRNA), a IncRNA, a tRNA, a shRNA, a self-amplifying mRNA (saRNA), a circular RNA, or combinations thereof. In some aspects, the RNA is a miRNA. In some aspects, the RNA is a siRNA. In some aspects, the RNA is a shRNA. In some aspects, the RNA is a saRNA. In some aspects, the small molecule comprises an impermeable small molecule.

In some aspects, the glial cell reprogramming factor comprises a transcription factor, and wherein the transcription factor is capable of reprogramming the one or more cells into a glial cell. In some aspects, the glial cell reprogramming factor comprises Yamanaka factors (OKSM) (e.g., Oct3/4, Sox2, c-Myc, Klf4), OLIG2, OLIG1, SOX5, SOX6, SOX8, SOX9, SOX10, MYRF, MYT1, NKX2.2, NKX6.1, NKX6.2, ZFP536, ASCL1, ST18, NFIA, ZEB2, or combinations thereof.

Also provided herein is a method of inducing the reprogramming of a cell into a glial cell comprising: passing a cell suspension, which comprises a population of cells, through a constriction under one or more parameters, wherein passing the cell suspension through the constriction under the one or more parameters deforms one or more cells of the population of cells, and thereby, causing a perturbation in the cell membrane of the one or more cells, and wherein the perturbation allows a glial cell reprogramming factor to enter the one or more cells, and thereby, induce the reprogramming of the one or more cells into a glial cell. In some aspects, the glial cell comprises an oligodendrocyte progenitor cell (OPC), oligodendrocyte, or both.

In some aspects, the above method comprises contacting the population of cells with the glial cell reprogramming factor prior to passing of the cell suspension through the constriction. In some aspects, the above method comprises contacting the population of cells with the glial cell reprogramming factor during the passing of the cell suspension through the constriction. In some aspects, the population of cells are contacted with the glial cell reprogramming factor during the passing of the cell suspension through the constriction. In some aspects, the above method comprises contacting the population of cells with the glial cell reprogramming factor after the passing of the cell suspension through the constriction. In some aspects, the population of cells are contacted with the glial cell reprogramming factor after the passing of the cell suspension through the constriction.

In some aspects, the glial cell reprogramming factor that can be used with the above methods comprises a nucleic acid, a polypeptide, a lipid, a carbohydrate, a small molecule, a metal-containing compound, an antibody, a transcription factor, a nanoparticle, a liposome, a fluorescently tagged molecule, or combinations thereof.

In some aspects, the nucleic acid comprises a DNA, RNA, or both. In some aspects, the DNA comprises a recombinant DNA, a cDNA, a genomic DNA, or combinations thereof. In some aspects, the RNA comprises a siRNA, a mRNA, a microRNA (miRNA), a IncRNA, a tRNA, a shRNA, a self-amplifying mRNA (saRNA), a circular RNA, or combinations thereof. In some aspects, the RNA is a miRNA. In some aspects, the RNA is a siRNA. In some aspects, the RNA is a shRNA. In some aspects, the RNA is a saRNA. In some aspects, the small molecule comprises an impermeable small molecule.

In some aspects, the glial cell reprogramming factor that can be used with the above methods comprises a transcription factor, and wherein the transcription factor is capable of reprogramming the one or more cells into a glial cell. In some aspects, the glial cell reprogramming factor comprises Yamanaka factors (OKSM) (e.g., Oct3/4, Sox2, c-Myc, Klf4), OLIG2, OLIG1, SOX5, SOX6, SOX8, SOX9, SOX10, MYRF, MYTI, NKX2.2, NKX6.1, NKX6.2, ZFP536, ASCL1, ST18, NFIA, ZEB2, or combinations thereof.

For any of the methods provided above, in some aspects, the one or more cells of the population of cells comprises stem cells, somatic cells, or both. In some aspects, the stem cells are hematopoietic stem cells (HSCs), induced pluripotent stem cells (iPSCs), embryonic stem cells, tissue-specific stem cells, mesenchymal stem cells, or combinations thereof. In some aspects, the stem cells are HSCs or iPSCs. In some aspects, the somatic cells comprise blood cells, fibroblasts, or both. In some aspects, the blood cells are PBMCs. In some aspects, the PBMCs comprise immune cells. In some aspects, the immune cells comprise a T cell, B cell, natural killer (NK) cell, dendritic cell (DC), NKT cell, mast cell, monocyte, macrophage, basophil, eosinophil, neutrophil, DC2.4 dendritic cell, or combinations thereof. In some aspects, the population of cells comprise T cells. In some aspects, the population of cells comprise monocytes.

For any of the methods provided above, in some aspects, the one or more parameters are selected from a cell density; pressure; length, width, and/or depth of the constriction; diameter of the constriction; diameter of the cells; temperature; entrance angle of the constriction; exit angle of the constriction; length, width, and/or width of an approach region; surface property of the constriction (e.g., roughness, chemical modification, hydrophilic, hydrophobic); operating flow speed; glial cell reprogramming factor concentration; viscosity, osmolarity, salt concentration, serum content, and/or pH of the cell suspension; time in the constriction; shear rate in the constriction; type of glial cell reprogramming factor, or combinations thereof.

In some aspects, the cell density is at least about 6×10⁷ cells/mL, at least about 7×10⁷ cells/mL, at least about 8×10⁷ cells/mL, at least about 9×10⁷ cells/mL, at least about 1×10⁸ cells/mL, at least about 1.1×10⁸ cells/mL, at least about 1.2×10⁸ cells/mL, at least about 1.3×10⁸ cells/mL, at least about 1.4×10⁸ cells/mL, at least about 1.5×10⁸ cells/mL, at least about 2.0×10⁸ cells/mL, at least about 3.0×10⁸ cells/mL, at least about 4.0×10⁸ cells/mL, at least about 5.0×10⁸ cells/mL, at least about 6.0×10⁸ cells/mL, at least about 7.0×10⁸ cells/mL, at least about 8.0×10⁸ cells/mL, at least about 9.0×10⁸ cells/mL, or at least about 1.0×10⁹ cells/mL or more. In some aspects, the pressure is at least about 30 psi, at least about 35 psi, at least about 40 psi, at least about 45 psi, at least about 50 psi, at least about 55 psi, at least about 60 psi, at least about 65 psi, at least about 70 psi, at least about 75 psi, at least about 80 psi, at least about 85 psi, at least about 90 psi, at least about 95 psi, at least about 100 psi, at least about 110 psi, at least about 120 psi, at least about 130 psi, at least about 140 psi, or at least about 150 psi.

In some aspects, the constriction is contained within a microfluidic chip. In some aspects, the diameter of the constriction is about 20%, about 30%, about 40%, about 50%, about 60%, about 70%, about 80%, about 90%, or about 99% of the diameter of the one or more cells of the population of cells. In some aspects, the length of the constriction is up to about 100 μm. In some aspects, the length of the constriction is less than about 1 μm. In some aspects, the length of the constriction is less than about 1 μm, less than about 5 μm, less than about 10 μm, less than about 20 μm, less than about 30 μm, less than about 40 μm, less than about 50 μm, less than about 60 μm, less than about 70 μm, less than about 80 μm, less than about 90 μm, or less than about 100 μm. In some aspects, the length of the constriction is about 1 μm, about 5 μm, about 10 μm, about 20 μm, about 30 μm, about 40 μm, about 50 μm, about 60 μm, about 70 μm, about 80 μm, about 90 μm, or about 100 μm. In some aspects, the width of the constriction is up to about 10 μm. In some aspects, the width of the constriction is less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm. In some aspects, the width of the constriction is between about 3 μm to about 10 μm. In some aspects, the width of the constriction is about 3 μm, about 4 μm, about 5 μm, about 6 μm, about 7 μm, about 8 μm, about 9 μm, or about 10 μm. In some aspects, the depth of the constriction is at least about 1 μm. In some aspects, the depth of the constriction is at least about 1 μm, at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm. In some aspects, the depth of the constriction is about 5 μm to about 90 μm. In some aspects, the depth is about 5 μm, about 10 μm, about 20 μm, about 30 μm, about 40 μm, about 50 μm, about 60 μm, about 70 μm, about 80 μm, or about 90 μm.

For any of the methods provided above, in some aspects, the population of cells are contacted with multiple glial cell reprogramming factors, such that at least two or more of the glial cell reprogramming factors are cable of entering the one or more cells after the perturbation of the cell membrane. In some aspects, the multiple glial cell reprogramming factors comprise at least about 2, at least about 3, at least about 4, at least about 5, at least about 6, at least about 7, at least about 8, at least about 9, or at least about 10 or more glial cell reprogramming factors. In some aspects, the multiple glial cell reprogramming factors are delivered into the cells concurrently. In some aspects, one or more of the glial cell reprogramming factors are delivered into the cells sequentially.

For any of the methods provided above, in some aspects, the method comprises passing the cell suspension through a plurality of constrictions. In some aspects, the plurality of constrictions comprise at least about 2, at least about 3, at least about 4, at least about 5, at least about 6, at least about 7, at least about 8, at least about 9, at least about 10, at least about 20, at least about 30, at least about 40, at least about 50, at least about 75, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1,000 or more separate constrictions.

In some aspects, each constriction of the plurality of constrictions are the same. In some aspects, one or more of the constrictions of the plurality of constrictions are different. In some aspects, one or more of the constrictions differ in their length, depth, width, or combinations thereof. In some aspects, each constriction of the plurality of constrictions is associated with the same glial cell reprogramming factor. In some aspects, one or more of the plurality of constrictions is associated with a different glial cell reprogramming factor.

In some aspects, the plurality of constrictions comprise a first constriction associated with a first glial cell reprogramming factor and a second constriction associated with a second glial cell reprogramming factor, wherein the cell suspension passes through the first constriction such that the first glial cell reprogramming factor is delivered to one or more cells of the plurality of cells, and then the cell suspension passes through the second constriction such that the second glial cell reprogramming factor is delivered to the one or more cells of the plurality of cells. In some aspects, the cell suspension is passed through the second constriction at least about 1 minute, at least about 30 minutes, at least about 1 hour, at least about 6 hours, at least about 12 hours, or at least about 1 day after the cell suspension is passed through the first constriction.

For any of the methods provided above, in some aspects, the method further comprises contacting the plurality of cells with an additional compound. In some aspects, the additional compound comprises a nucleic acid, a polypeptide, a lipid, a carbohydrate, a small molecule, a metal-containing compound, an antibody, a transcription factor, a nanoparticle, a liposome, a fluorescently tagged molecule, or combinations thereof. In some aspects, the contacting of the additional compound with the plurality of cells occurs concurrently with the glial cell reprogramming factor. In some aspects, the contacting of the additional compound with the plurality of cells occurs prior to or after the contacting of the plurality of cells with the glial cell reprogramming factor.

In some aspects, the additional compound is a signaling molecule that is capable of inducing the formation of a glial cell. In some aspects, the signaling molecule comprises a SHH, bFGF, PDGF, cAMP pathway activator (e.g., forskolin), TGF-β blocker (e.g., SB431542), BMP blocker (dorsomorphin), retinoic acid, rock-inhibitor (e.g., Y26732), insulin growth factor 1, ascorbic acid, neurotropic factors, or combinations thereof. In some aspects, the neurotrophic factors comprise a brain-derived neurotrophic factor (BDNF), glia-derived neurotrophic factor (GDNF), nerve growth factor (NGF), and cerebral dopamine neurotrophic factor (CDNF), or combinations thereof.

In some aspects, the above method comprising contacting the plurality of cells with the additional compound comprises collecting the cell suspension that passed through the constriction and culturing the cell suspension in the presence of the signaling molecule. In some aspects, the cell suspension is cultured in the presence of the signaling molecule for at least about one day, at least about two days, at least about three days, at least about four days, at least about five days, at least about six days, at least about one week, at least about two weeks, at least about three weeks, or at least about four weeks.

In some aspects, the additional compound is a nucleic acid encoding an enzyme that confers resistance to an antibiotic. In some aspects, the method comprising contacting the plurality of cells with the nucleic acid encoding an enzyme that confers resistance to an antibiotic comprises collecting the cell suspension that passed through the constriction and treating the cell suspension with the antibiotic. In some aspects, the enzyme comprises puromycin-N-acetyltransferase and the antibiotic is puromycin. In some aspects, after the treatment with the antibiotic, the proportion of glial cells present in the cell suspension is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold.

Provided herein is a composition comprising a population of differentiated cells produced by any of the methods provided herein. Also provided herein is a composition comprising a population of cells and a glial cell reprogramming factor under one or more parameters resulting in deformation of one or more cells of the population of cells, wherein the one or more cells comprise a perturbation in the cell membrane sufficient to allow the glial cell reprogramming factor to enter the one or more cells.

In some aspects, one or more cells of the population of cells of the composition comprises stem cells, somatic cells, or both. In some aspects, the stem cells are hematopoietic stem cells (HSCs), induced pluripotent stem cells (iPSCs), embryonic stem cells, tissue-specific stem cells, mesenchymal stem cells, or combinations thereof. In some aspects, the stem cells are HSCs or iPSCs. In some aspects, the somatic cells comprise blood cells, fibroblasts, or both. In some aspects, the blood cells are PBMCs. In some aspects, the PBMCs comprise immune cells. In some aspects, the immune cells comprise a T cell, B cell, natural killer (NK) cell, dendritic cell (DC), NKT cell, mast cell, monocyte, macrophage, basophil, eosinophil, neutrophil, DC2.4 dendritic cell, or combinations thereof. In some aspects, the population of cells comprise T cells. In some aspects, the population of cells comprise monocytes.

For any of the compositions provided above, in some aspects, the glial cell reprogramming factor comprises a nucleic acid, a polypeptide, a lipid, a carbohydrate, a small molecule, a metal-containing compound, an antibody, a transcription factor, a nanoparticle, a liposome, a fluorescently tagged molecule, or combinations thereof. In some aspects, the nucleic acid comprises a DNA, RNA, or both. In some aspects, the DNA comprises a recombinant DNA, a cDNA, a genomic DNA, or combinations thereof. In some aspects, the RNA comprises a siRNA, a mRNA, a microRNA (miRNA), a IncRNA, a tRNA, a shRNA, a self-amplifying mRNA (saRNA), a circular RNA, or combinations thereof. In some aspects, the RNA is a miRNA. In some aspects, the RNA is a siRNA. In some aspects, the RNA is a shRNA. In some aspects, the RNA is a saRNA. In some aspects, the small molecule comprises an impermeable small molecule.

For any of the compositions provided above, in some aspects, the one or more parameters are selected from a cell density; a pressure; a length, width, and/or depth of the constriction; a diameter of the constriction; a diameter of the cells; a temperature; an entrance angle of the constriction; an exit angle of the constriction; a length, width, and/or width of an approach region; a surface property of the constriction (e.g., roughness, chemical modification, hydrophilic, hydrophobic); an operating flow speed; a glial cell reprogramming factor concentration; a viscosity, osmolarity, salt concentration, serum content, and/or pH of the cell suspension; time in the constriction; shear rate in the constriction; type of glial cell reprogramming factor or combinations thereof.

In some aspects, the cell density is at least about 6×10⁷ cells/mL, at least about 7×10⁷ cells/mL, at least about 8×10⁷ cells/mL, at least about 9×10⁷ cells/mL, at least about 1×10⁸ cells/mL, at least about 1.1×10⁸ cells/mL, at least about 1.2×10⁸ cells/mL, at least about 1.3×10⁸ cells/mL, at least about 1.4×10⁸ cells/mL, at least about 1.5×10⁸ cells/mL, at least about 2.0×10⁸ cells/mL, at least about 3.0×10⁸ cells/mL, at least about 4.0×10⁸ cells/mL, at least about 5.0×10⁸ cells/mL, at least about 6.0×10⁸ cells/mL, at least about 7.0×10⁸ cells/mL, at least about 8.0×10⁸ cells/mL, at least about 9.0×10⁸ cells/mL, or at least about 1.0×10⁹ cells/mL or more. In some aspects, the pressure is at least about 30 psi, at least about 35 psi, at least about 40 psi, at least about 45 psi, at least about 50 psi, at least about 55 psi, at least about 60 psi, at least about 65 psi, at least about 70 psi, at least about 75 psi, at least about 80 psi, at least about 85 psi, at least about 90 psi, at least about 95 psi, at least about 100 psi, at least about 110 psi, at least about 120 psi, at least about 130 psi, at least about 140 psi, or at least about 150 psi.

In some aspects, the constriction is contained within a microfluidic chip. In some aspects, the diameter of the constriction is about 20%, about 30%, about 40%, about 50%, about 60%, about 70%, about 80%, about 90%, or about 99% of the diameter of the one or more cells of the population of cells. In some aspects, the length of the constriction is up to about 100 μ. In some aspects, the length of the constriction is less than about 1 μm. In some aspects, the length of the constriction is less than about 1 μm, less than about 5 μm, less than about 10 μm, less than about 20 μm, less than about 30 μm, less than about 40 μm, less than about 50 μm, less than about 60 μm, less than about 70 μm, less than about 80 μm, less than about 90 μm, or less than about 100 μm. In some aspects, the length of the constriction is about 1 μm, about 5 μm, about 10 μm, about 20 μm, about 30 μm, about 40 μm, about 50 μm, about 60 μm, about 70 μm, about 80 μm, about 90 μm, or about 100 μm. In some aspects, the width of the constriction is up to about 10 μm. In some aspects, the width of the constriction is less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm. In some aspects, the width of the constriction is between about 3 μm to about 10 μm. In some aspects, the width of the constriction is about 3 μm, about 4 μm, about 5 μm, about 6 μm, about 7 μm, about 8 μm, about 9 μm, or about 10 μm. In some aspects, the depth of the constriction is at least about 1 μm. In some aspects, the depth of the constriction is at least about 1 μm, at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm. In some aspects, the depth of the constriction is about 5 μm to about 90 μm. In some aspects, the depth is about 5 μm, about 10 μm, about 20 μm, about 30 μm, about 40 μm, about 50 μm, about 60 μm, about 70 μm, about 80 μm, or about 90 μm.

Present disclosure further provides a cell comprising a perturbation in the cell membrane due to one or more parameters which deform the cell thereby causing the perturbation in the cell membrane of the cell such that a glial cell reprogramming factor can enter the cell. Also provided herein is a cell comprising a glial cell reprogramming factor, wherein the glial cell reprogramming factor entered the cell through a perturbation in the cell membrane due to one or more parameters which deformed the cell thereby causing the perturbation in the cell membrane of the cell such that the glial cell reprogramming factor enters the cell.

In some aspects, the cell comprises stem cells, somatic cells, or both. In some aspects, the stem cells are hematopoietic stem cells (HSCs), induced pluripotent stem cells (iPSCs), embryonic stem cells, tissue-specific stem cells, mesenchymal stem cells, or combinations thereof. In some aspects, the stem cells are HSCs or iPSCs. In some aspects, the somatic cells comprise blood cells, fibroblasts, or both. In some aspects, the blood cells are PBMCs. In some aspects, the PBMCs comprise immune cells. In some aspects, the immune cells comprise a T cell, B cell, natural killer (NK) cell, dendritic cell (DC), NKT cell, mast cell, monocyte, macrophage, basophil, eosinophil, neutrophil, DC2.4 dendritic cell, or combinations thereof. In some aspects, the population of cells comprise T cells. In some aspects, the population of cells comprise monocytes.

For any of the cells provided above, in some aspects, the glial cell reprogramming factor comprises a nucleic acid, a polypeptide, a lipid, a carbohydrate, a small molecule, a metal-containing compound, an antibody, a transcription factor, a nanoparticle, a liposome, a fluorescently tagged molecule, or combinations thereof.

In some aspects, the nucleic acid comprises a DNA, RNA, or both. In some aspects, the DNA comprises a recombinant DNA, a cDNA, a genomic DNA, or combinations thereof. In some aspects, the RNA comprises a siRNA, a mRNA, a microRNA (miRNA), a IncRNA, a tRNA, a shRNA, a self-amplifying mRNA (saRNA), a circular RNA, or combinations thereof. In some aspects, the RNA is a miRNA. In some aspects, the RNA is a siRNA. In some aspects, the RNA is a shRNA. In some aspects, the RNA is a saRNA. In some aspects, the small molecule comprises an impermeable small molecule.

For any of the cells provided above, in some aspects, the one or more parameters are selected from a cell density; pressure; length, width, and/or depth of the constriction; diameter of the constriction; diameter of the cells; temperature; entrance angle of the constriction; exit angle of the constriction; length, width, and/or width of an approach region; surface property of the constriction (e.g., roughness, chemical modification, hydrophilic, hydrophobic); operating flow speed; glial cell reprogramming factor concentration; viscosity, osmolarity, salt concentration, serum content, and/or pH of the cell suspension; time in the constriction; shear rate in the constriction; type of glial cell reprogramming factor, or combinations thereof.

In some aspects, the cell density is at least about 6×10⁷ cells/mL, at least about 7×10⁷ cells/mL, at least about 8×10⁷ cells/mL, at least about 9×10⁷ cells/mL, at least about 1×10⁸ cells/mL, at least about 1.1×10⁸ cells/mL, at least about 1.2×10⁸ cells/mL, at least about 1.3×10⁸ cells/mL, at least about 1.4×10⁸ cells/mL, at least about 1.5×10⁸ cells/mL, at least about 2.0×10⁸ cells/mL, at least about 3.0×10⁸ cells/mL, at least about 4.0×10⁸ cells/mL, at least about 5.0×10⁸ cells/mL, at least about 6.0×10⁸ cells/mL, at least about 7.0×10⁸ cells/mL, at least about 8.0×10⁸ cells/mL, at least about 9.0×10⁸ cells/mL, or at least about 1.0×10⁹ cells/mL or more. In some aspects, the pressure is at least about 30 psi, at least about 35 psi, at least about 40 psi, at least about 45 psi, at least about 50 psi, at least about 55 psi, at least about 60 psi, at least about 65 psi, at least about 70 psi, at least about 75 psi, at least about 80 psi, at least about 85 psi, at least about 90 psi, at least about 95 psi, at least about 100 psi, at least about 110 psi, at least about 120 psi, at least about 130 psi, at least about 140 psi, or at least about 150 psi.

In some aspects, the constriction is contained within a microfluidic chip. In some aspects, the diameter of the constriction is about 20%, about 30%, about 40%, about 50%, about 60%, about 70%, about 80%, about 90%, or about 99% of the diameter of the one or more cells of the population of cells. In some aspects, the length of the constriction is up to about 100 μm. In some aspects, the length of the constriction is less than about 1 μm. In some aspects, the length of the constriction is less than about 1 μm, less than about 5 μm, less than about 10 μm, less than about 20 μm, less than about 30 μm, less than about 40 μm, less than about 50 μm, less than about 60 μm, less than about 70 μm, less than about 80 μm, less than about 90 μm, or less than about 100 μm. In some aspects, the length of the constriction is about 1 μm, about 5 μm, about 10 μm, about 20 μm, about 30 μm, about 40 μm, about 50 μm, about 60 μm, about 70 μm, about 80 μm, about 90 μm, or about 100 μm. In some aspects, the width of the constriction is up to about 10 μm. In some aspects, the width of the constriction is less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm. In some aspects, the width of the constriction is between about 3 μm to about 10 μm. In some aspects, the width of the constriction is about 3 μm, about 4 μm, about 5 μm, about 6 μm, about 7 μm, about 8 μm, about 9 μm, or about 10 μm. In some aspects, the depth of the constriction is at least about 1 μm. In some aspects, the depth of the constriction is at least about 1 μm, at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm. In some aspects, the depth of the constriction is about 5 μm to about 90 μm. In some aspects, the depth is about 5 μm, about 10 μm, about 20 μm, about 30 μm, about 40 μm, about 50 μm, about 60 μm, about 70 μm, about 80 μm, or about 90 μm.

Provided herein is a method of treating a demyelinating disease in a subject in need thereof, comprising administering to the subject a plurality of glial cells, wherein the glial cells are produced by any of the methods provided herein. In some aspects, the glial cells comprise oligodendrocyte progenitor cells, oligodendrocytes, or both. In some aspects, after the administration, the glial cells differentiate into oligodendrocytes. In some aspects, the oligodendrocytes remyelinate one or more neuronal axons within the subject.

Also provided herein is a method of treating a demyelinating disease in a subject in need thereof, comprising administering to the subject any of the compositions or cells described herein.

For any of the treatment methods provided above, in some aspects, the demyelinating disease comprises a multiple sclerosis, acute disseminated encephalomyelitis (ADEM), acute hemorrhagic leukoencephalitis, acute transverse myelitis, adrenoleukodystrophy, adrenomyeloneuropathy, Alexander disease, Alzheimer's disease, aminoacidurias, amyotrophic lateral sclerosis, anti-MAG peripheral neuropathy, anti-MOG associated spectrum, Balo concentric sclerosis, brain injury, CAMFAK Syndrome, Canavan disease, carbon monoxide toxicity, central pontine myelinolysis, cerebral hypoxia, cerebral ischemia, Charcot-Marie-Tooth disease, chronic inflammatory demyelinating polyneuropathy, chronic traumatic encephalopathy, clinically isolated syndrome (CIS), congenital cataract, copper deficiency associated condition, delayed post-hypoxic leukoencephalopathy, diffuse cerebral sclerosis of Schilder, diffuse myelinoclastic sclerosis, extrapontine myelinolysis Gaucher disease, Guillain-Barré syndrome, hereditary neuropathy, hereditary neuropathy with liability to pressure palsy, HTLV-1-associated myelopathy, Hurler syndrome, hypomyelination, hypoxic brain injury, Krabbe disease, Leber hereditary optic atrophy and related mitochondrial disorders, leukodystrophic disorders, Marchiafava-Bignami disease, metachromatic leukodystrophy, multiple sclerosis, multiple system atrophy, myelinoclastic disorders, myelopathy, nerve injury, neuromyelitis optica (NMO), Niemann-Pick disease, optic neuropathy, optic neuritis (e.g., acute optic neuritis and chronic relapsing inflammatory optic neuritis (CRION)), osmotic semyelination syndrome, Parkinson's disease, Pelizaeus-Merzbacher disease, peripheral neuropathy, phenylketonuria, progressive inflammatory neuropathy, progressive multifocal leukoencephalopathy, progressive subcortical ischemic demyelination, reperfusion injury, Schilder disease, solitary sclerosis, spinal cord injury, subacute sclerosing panencephalitis, Tabes dorsalis, Tay-Sachs disease, traumatic brain injury, tropical spastic paraparesis, vitamin B12 deficiency, or a combination thereof.

In some aspects, the multiple sclerosis comprises a clinically isolated syndrome (“CIS”), relapsing-remitting MS (“RRMS”), secondary progressive MS (“SPMS”), primary progressive MS (“PPMS”), or a combination thereof.

Provided herein is a method of promoting the remyelination of demyelinated neuronal axons in a subject in need thereof comprising administering to the subject a population of glial cells, wherein the glial cells are produced according to any of the methods provided herein, and wherein after the administration, one or more of the demyelinated neuronal axons are remyelinated. In some aspects, the glial cells comprise oligodendrocyte progenitor cells, oligodendrocytes, or both.

Also provided herein is a method of promoting the remyelination of demyelinated neuronal axons in a subject in need thereof comprising administering to the subject any of the compositions or cells described herein.

Provided herein is a method of reducing the demyelination of a myelinated neuronal axon in a subject in need thereof comprising administering to the subject a population of glial cells, wherein the glial cells are produced according to any of the methods provided herein. In some aspects, the glial cells comprise oligodendrocyte progenitor cells, oligodendrocytes, or both.

Provided herein is a method of reducing the demyelination of a myelinated neuronal axon in a subject in need thereof comprising administering to the subject any of the compositions or cells provided herein.

### DETAILED DESCRIPTION OF DISCLOSURE

The present disclosure is generally directed to methods of producing glial cells (e.g., oligodendrocyte progenitor cells) and the use of such cells to treat demyelinating diseases (e.g., multiple sclerosis). More particularly, the methods provided herein comprise passing a cell suspension comprising a population of cells (e.g., PBMCs or a subset therein (e.g., T cells or monocytes); or stem cells (e.g., HSCs)) through a constriction under one or more parameters, resulting in the transient formation of perturbations within the cell membrane of the cells. In some aspects, the methods further comprise one or more glial cell reprogramming factors, which are capable of entering the cells through the perturbations and thereby, inducing the cells to become oligodendrocytes. Non-limiting examples of the various aspects are shown in the present disclosure.

### I. General Techniques

Some of the techniques and procedures described or referenced herein are generally well understood and commonly employed using conventional methodology by those skilled in the art, such as, for example, the widely utilized methodologies described in Molecular Cloning: A Laboratory Manual (Sambrook et al., 4^{th} ed., Cold Spring Harbor Laboratory Press, Cold Spring Harbor, N.Y., 2012); Current Protocols in Molecular Biology (F.M. Ausubel, et al. eds., 2003); the series Methods in Enzymology (Academic Press, Inc.); PCR 2: A Practical Approach (M.J. MacPherson, B.D. Hames and G.R. Taylor eds., 1995); Antibodies, A Laboratory Manual (Harlow and Lane, eds., 1988); Culture of Animal Cells: A Manual of Basic Technique and Specialized Applications (R.I. Freshney, 6^{th} ed., J. Wiley and Sons, 2010); Oligonucleotide Synthesis (M.J. Gait, ed., 1984); Methods in Molecular Biology, Humana Press; Cell Biology: A Laboratory Notebook (J.E. Cellis, ed., Academic Press, 1998); Introduction to Cell and Tissue Culture (J.P. Mather and P.E. Roberts, Plenum Press, 1998); Cell and Tissue Culture: Laboratory Procedures (A. Doyle, J.B. Griffiths, and D.G. Newell, eds., J. Wiley and Sons, 1993-8); Handbook of Experimental Immunology (D.M. Weir and C.C. Blackwell, eds., 1996); Gene Transfer Vectors for Mammalian Cells (J.M. Miller and M.P. Calos, eds., 1987); PCR: The Polymerase Chain Reaction, (Mullis et al., eds., 1994); Current Protocols in Immunology (J.E. Coligan et al., eds., 1991); Short Protocols in Molecular Biology (Ausubel et al., eds., J. Wiley and Sons, 2002); Immunobiology (C.A. Janeway et al., 2004); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer: Principles and Practice of Oncology (V.T. DeVita et al., eds., J.B. Lippincott Company, 2011).

### II. Definitions

For purposes of interpreting this specification, the following definitions will apply and whenever appropriate, terms used in the singular will also include the plural and vice versa. In the event that any definition set forth below conflicts with any document incorporated herein by reference, the definition set forth herein shall control. Additional definitions are set forth throughout the detailed description.

As used herein, the singular form term “a,” “an,” and “the” entity refers to one or more of that entity unless indicated otherwise. As such, the terms “a” (or “an” or “the”), “one or more,” and “at least one” can be used interchangeably herein.

It is understood that aspects and aspects of the disclosure described herein include “comprising,” “consisting,” and “consisting essentially of” aspects and aspects. It is also understood that wherever aspects and aspects are described herein with the language “comprising,” otherwise analogous aspects or aspects described in terms of “consisting of” and/or “consisting essentially of” are also provided.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

For all compositions described herein, and all methods using a composition described herein, the compositions can either comprise the listed components or steps, or can “consist essentially of” the listed components or steps. When a composition is described as “consisting essentially of” the listed components, the composition contains the components listed, and can further contain other components which do not substantially affect the methods disclosed, but do not contain any other components which substantially affect the methods disclosed other than those components expressly listed; or, if the composition does contain extra components other than those listed which substantially affect the methods disclosed, the composition does not contain a sufficient concentration or amount of the extra components to substantially affect the methods disclosed. When a method is described as “consisting essentially of” the listed steps, the method contains the steps listed, and can further contain other steps that do not substantially affect the methods disclosed, but the method does not contain any other steps which substantially affect the methods disclosed other than those steps expressly listed. As a non-limiting specific example, when a composition is described as “consisting essentially of” a component, the composition can additionally contain any amount of pharmaceutically acceptable carriers, vehicles, or diluents and other such components which do not substantially affect the methods disclosed.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “constriction” as used herein refers to a narrowed passageway. In some aspects, the constriction is a microfluidic channel, such as that contained within a microfluidic device. In some aspects, the constriction is a pore or contained within a pore. Where the constriction is a pore, in some aspects, the pore is contained in a surface. Unless indicated otherwise, the term constriction refers to both microfluidic channels and pores, as well as other suitable constrictions available in the art. Therefore, where applicable, disclosures relating to microfluidic channels can also apply to pores and/or other suitable constrictions available in the art. Similarly, where applicable, disclosures relating to pores can equally apply to microfluidic channels and/or other suitable constrictions available in the art.

The term “pore” as used herein refers to an opening, including without limitation, a hole, tear, cavity, aperture, break, gap, or perforation within a material. In some aspects, (where indicated) the term refers to a pore within a surface of a microfluidic device, such as those described in the present disclosure. In some aspects, (where indicated) a pore can refer to a pore in a cell wall and/or cell membrane.

The term “membrane” as used herein refers to a selective barrier or sheet containing pores. The term includes, but is not limited to, a pliable sheet-like structure that acts as a boundary or lining. In some aspects, the term refers to a surface or filter containing pores. This term is distinct from the term “cell membrane,” which refers to a semipermeable membrane surrounding the cytoplasm of cells.

The term “filter” as used herein refers to a porous article that allows selective passage through the pores. In some aspects, the term refers to a surface or membrane containing pores.

As used herein, the terms “deform” and “deformity” (including derivatives thereof) refer to a physical change in a cell. As described herein, as a cell passes through a constriction (such as those of the present disclosure), it experiences various forces due to the constraining physical environment, including but not limited to mechanical deforming forces and/or shear forces that causes perturbations in the cell membrane. As used herein, a “perturbation” within the cell membrane refers to any opening in the cell membrane that is not present under normal steady state conditions (e.g., no deformation force applied to the cells). Perturbation can comprise a hole, tear, cavity, aperture, pore, break, gap, perforation, or combinations thereof.

The term “heterogeneous” as used herein refers to something which is mixed or not uniform in structure or composition. In some aspects, the term refers to pores having varied sizes, shapes, or distributions within a given surface.

The term “homogeneous” as used herein refers to something which is consistent or uniform in structure or composition throughout. In some aspects, the term refers to pores having consistent sizes, shapes, or distribution within a given surface.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as can typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P—NH2) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “polypeptide” and “protein” are used interchangeably to refer to a polymer of amino acid residues, and are not limited to a minimum length. Such polymers of amino acid residues can contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications can be deliberate, as through site-directed mutagenesis, or can be accidental, such as through mutations of hosts which produce the proteins or errors due to PCR amplification.

As used herein, the term “demyelinating disease” refers to any disorder of the nervous system in which there is reduced myelination, including, but not limited to, (i) disorders in which insufficient or dysfunctional myelin is generated during development (e.g., hypermyelination), and (ii) disorders in which the myelin sheath of neurons is damaged. In some aspects, the neuronal axon of a subject suffering from or at risk of developing a demyelinating disease is completely demyelinated. In some aspects, the neuronal axon of a subject suffering from or at risk of developing a demyelinating disease is partially demyelinated (e.g., at least 5%, at least 10%, at least 20%, at least 30%, at least 40%, at least 50%, at least 60%, at least 70%, at least 80%, or at least 95% compared to a reference neuronal axon that is fully myelinated). Accordingly, unless indicated otherwise, the term “demyelination” comprises both complete demyelination and partial demyelination. Non-limiting examples of demyelinating diseases are provided elsewhere in the present disclosure.

As used herein, the term “hypomyelination” refers to a deficiency in myelin for any reason (e.g., body is unable to produce myelin at normal levels). Unless indicated otherwise, hypomyelination includes demyelination (related to myelin destruction) and “dysmyelination” (related to abnormal myelin deposition, e.g., due to dysfunctions of the oligodendrocytes). Accordingly, hypomyelination includes diseases in which insufficient myelin is generated during development as well as diseases associated with demyelination and/or dysmyelination.

“Myelin” and “myelin sheath” refer to the specialized membrane formed by oligodendrocytes that insulates the axons of neurons. The insulation provided by the myelin sheath helps to increase the rate of transmission of nerve signals along the axon, thereby promoting timely and energetically efficient neuronal signaling. Myelin also provides metabolic support of the axons, maintaining their health and survival.

As used herein, the term “glial cells” (also known as “glia,” “gliocyte,” and “neuroglia”) refers to non-neuronal cells present within the central nervous system (brain and spinal cord) and the peripheral nervous system. Glial cells do not produce electrical impulses and instead largely provide structural and metabolic support for neurons. As used herein, glial cells comprise oligodendrocyte progenitor cells (OPCs), oligodendrocytes, astrocytes, ependymal cells, microglia, or a combination thereof. In some aspects, glial cells that can be produced using the methods provided herein comprise OPCs.

As used herein, the term “oligodendrocytes” (also known as “oligodendroglia”) refer to a type of neuroglia whose main functions are to provide support and insulation to axons in the central nervous system of many vertebrates (e.g., humans). As used herein, the term “oligodendrocyte progenitor cells” or “OPCs” (also known in the art as “oligodendrocyte precursor cells,” “polydendrocytes,” “NG2 cells,” “NG2 glia,” and “O-2A cells”) refer to a subtype of glial cells in the central nervous system, which traditionally give rise to the oligodendrocytes during development. Various markers can be used to determine the differentiation status of glial cells. Non-limiting examples of markers that can be used to specifically assess the degree of OPC differentiation include: A2B5 (a cell surface protein epitope characteristic of bipotential glial progenitor cells able to differentiate into astrocytes or oligodendrocytes), NG2 and PDGFR-a (for proliferative OPCs), O4 (for early pre-myelinating state), PLP and CNPase (for late-stage myelinating OPCs), APC, MOG, and MBP (for mature OLs), and endogenous Olig2 (as pan-OL lineage marker). In some aspects, the above markers for OPC differentiation can be used alone or in combination with markers for other cell types, e.g., CD271 or PMP22 (for Schwann cells), TUJ1 (for neurons), PAX6 and NESTIN (for common neural stem/progenitor cells), GFAP (for astroglia and neural progenitors), CD11b and IBA1 (for microglia), POU5F1 (for pluripotent stem cells). The expression of such markers can be assessed using any suitable methods known in the art (e.g., quantitative PCR, immunocytochemistry, and flow cytometry).

As used herein, the term “remyelination” (or derivatives thereof) refers to generation of new myelin sheaths around demyelinated (e.g., including partially myelinated) axons. The remyelination process traditionally involves the differentiation of OPCs into oligodendrocytes that generate functional myelin sheaths around demyelinated axons. Remyelination of the axons can restore action potential conduction properties to axons, and thereby, promote and/or improve neurological function. Further, remyelination can provide metabolic support to axons, preventing their damage or loss. In the context of the present application, unless otherwise specified, “remyelination” refers to any aspect of a process that can result in remyelination (e.g., generation of myelin sheaths by oligodendrocytes around demyelinated axons, migration or colonization of OPCs to sites of demyelinated axons, and/or differentiation of OPCs into oligodendrocytes).

As used herein, the term “subject” refers to any animal subject including a human, a laboratory animal (e.g., a non-human primate, rat, and mouse), livestock (e.g., cow, sheep, goat, pig, turkey, and chicken), and household pets (e.g., dog, cat, and rodent).

The terms “treat,” “treating,” and “treatment,” as used herein, refer to any type of intervention or process performed on, or administering an active agent (e.g., oligodendrocytes produced according to the methods of the present disclosure) to, the subject with the objective of reversing, alleviating, ameliorating, inhibiting, or slowing down; or preventing the progression, development, severity or recurrence of a symptom, complication, condition or one or more biochemical indicia associated with a disease, or enhancing overall survival. As described herein, in some aspects, treatment can be of a subject having a disease (e.g., exhibiting one or more symptoms associated with the disease). In some aspects, treatment can be of a subject with some degree of demyelination but not yet exhibiting any symptoms associated with the disease. In the context of such subjects, administering an oligodendrocyte produced as described herein can help delay or prevent the onset of symptoms associated with the disease.

As used herein, the terms “ug” and “uM” are used interchangeably with “μg” and “μM,” respectively.

Various aspects described herein are described in further detail in the following subsections. The various aspects and embodiments of the invention disclosed throughout the specification can optionally be combined.

### III. Methods of the Disclosure

In some aspects, the present disclosure relates to methods of delivering a cargo (also referred to herein as “payload”) that is capable of reprogramming a cell into glial cells (e.g., glial cell reprogramming factors described herein) by passing the cells through a constriction (such as those described herein). As demonstrated herein, as the cells pass through the constriction, they become transiently deformed, such that cell membrane of the cells is perturbed. The perturbations within the cell membrane can allow various payloads to enter or loaded into the cell (e.g., through diffusion). As further described herein, in some aspects, the glial cells which can be produced using the present disclosure are OPCs. Not to be bound by any one theory, in some aspects, such OPCs, when administered to a subject, can further differentiate into oligodendrocytes, resulting in remyelination of one or more neuronal axons within the subject. The specific process by which the cells pass through a constriction and become transiently deformed is referred to herein as “squeeze processing” or “squeezing.”

As further described herein, the squeeze processing methods of the present disclosure have certain distinct properties that are not shared by other delivery methods known in the art. For example, in addition to the improved ability to deliver various types of payloads into a cell, the squeeze processing methods described herein exert minimal lasting effects on the cells. Compared to traditional delivery methods such as electroporation, the squeeze processing methods of the present disclosure preserve both the structural and functional integrity of the squeezed cells. Contrary to the delivery methods provided herein, electroporation can induce broad and lasting alterations in gene expression, which can lead to non-desired effects on the cells (e.g., non-specific activation of cells). With the present methods, any alterations to the cells (e.g., perturbations in the cell membrane) is transient and quickly repaired once the cells are removed from the constriction.

As demonstrated herein, through such methods, different aspects of a cell can be modified. In particular, by using a payload that is capable of reprogramming a cell into glial cells (i.e., “glial cell reprogramming factor”; also referred to herein as “glial cell differentiation factor”), the squeeze processing methods provided herein can be used to specifically produce different glial cells. For example, in some aspects, by delivering an OPC reprogramming factor to a cell using squeeze processing, the methods provided herein can be used to specifically produce OPCs. As is apparent from the present disclosure “OPC reprogramming factor” refers to a type of glial cell reprogramming factor that is capable of reprogramming cells specifically into oligodendrocyte progenitor cells. Unless indicated otherwise, the term “OPC reprogramming factor” and “glial cell reprogramming factor” are used interchangeably. Non-limiting examples of such reprogramming factors are provided elsewhere in the present disclosure. While the present disclosure generally discloses the use of glial cell reprogramming factors, it will be apparent to those skilled in the art that the disclosures provided herein could also apply to other types of payloads (e.g., those that enhance one or other properties of the oligodendrocytes produced using the methods provided herein).

Accordingly, in some aspects, provided herein is a method of producing glial cells (e.g., OPCs), wherein the method comprises passing a cell suspension through a constriction under one or more parameters, wherein the cell suspension comprises a plurality of cells (e.g., PBMCs, e.g., T cells or monocytes; or stem cells, e.g., HSCs), wherein one or more cells of the plurality of cells become transiently deformed as they pass through the constriction, resulting in perturbations in the cell membrane of the one or more cells, which allow a glial cell reprogramming factor to enter the cells and thereby, reprogram the cells to become glial cells (e.g., OPCs).

In some aspects, the above method further comprises contacting the population of cells with the glial cell reprogramming factor prior to passing the cell suspension through the constriction. For instance, in some aspects, prior to passing the cell suspension through the constriction, the method comprises contacting the population of cells with a glial cell reprogramming factor to produce the cell suspension (e.g., the population of cells and glial cell reprogramming factor are mixed together in a single cell suspension). As is apparent from the present disclosure, in some aspects, contacting the population of cells with the glial cell reprogramming factor prior to the squeezing can help with delivery efficiency, as the glial cell reprogramming factor would be able to enter the cell as soon as the perturbations in the cell membrane are created through the squeeze processing.

In some aspects, a method of producing glial cells (e.g., OPCs) described herein comprises contacting the population of cells with the glial cell reprogramming factor as the cells pass through the constriction. In some aspects, the population of cells are first contacted with the glial cell reprogramming factor during the passing of the cell suspension through the constriction. In some aspects, the population of cells are in contact with the glial cell reprogramming factor both prior to the passing step (i.e., passing of the cell suspension through the constriction) and during the passing step.

In some aspects, the method comprises contacting the population of cells with the glial cell reprogramming factor after the passing of the cell suspension through the constriction. In some aspects, the population of cells are first contacted with the glial cell reprogramming factor after the passing of the cell suspension through the constriction. In some aspects, the population of cells are in contact with the glial cell reprogramming factor prior to, during, and/or, after the passing step. As further described elsewhere in the present disclosure, when the population of cells are contacted with the glial cell reprogramming factor after the passing step, the contacting occurs soon after the cells have passed through the constriction, such that there are still perturbations within the cell membrane.

As further described elsewhere in the present disclosure, the methods provided herein can comprise multiple squeeze processing steps. For example, in some aspects, multiple squeeze processing steps can be used to repeatedly deliver a glial cell reprogramming factor (or a combination of reprogramming factors) to a cell (e.g., daily). In some aspects, multiple squeeze processing steps can be used to deliver a first glial cell reprogramming factor (or a first combination of reprogramming factors) using a first squeeze processing step (e.g., on day 0) and a second glial cell reprogramming factor (or a second combination of reprogramming factors) using a second squeeze processing step (e.g., on day 1).

As used herein, the “contacting” that can occur between a cell and a glial cell reprogramming factor includes that a cell can be in contact with the glial cell reprogramming factor as long as the glial reprogramming factor is capable of entering the cell once there are perturbations within the cell membrane of the cell. To help illustrate, in some aspects, a cell and a glial cell reprogramming factor are in contact if they are both present within the same cell suspension.

### III.A. Cell Suspensions

In some aspects, a cell suspension described herein comprises any suitable cells known in the art that can be modified (e.g., by introducing a glial cell reprogramming factor) using the squeeze processing methods described herein, and programmed to become glial cells (e.g., OPCs).

In some aspects, the cells which are useful for the present disclosure are stem cells. As used herein, the term “stem cells” refer to cells having not only self-replication ability but also the ability to differentiate into other types of cells (e.g., glial cells, e.g., OPCs). In some aspects, stem cells useful for the present disclosure comprise induced pluripotent stem cells (iPSCs), embryonic stem cells (ESCs), tissue-specific stem cells (e.g., liver stem cells, cardiac stem cells, or neural stem cells), mesenchymal stem cells, hematopoietic stem cells (HSCs), or combinations thereof. In some aspects, the stem cells are iPSCs. In some aspects, the stem cells are HSCs.

In some aspects, the cells are somatic cells. As used herein, the term “somatic cells” refer to any cell in the body that are not gametes (sperm or egg), germ cells (cells that go on to become gametes), or stem cells. Non-limiting examples of somatic cells include blood cells, bone cells, muscle cells, nerve cells, or combinations thereof. In some aspects, somatic cells useful for the present disclosure comprise blood cells. In some aspects, the blood cells are peripheral blood mononuclear cells (PBMCs). As used herein, “PBMCs” refer to any peripheral blood cells having a round nucleus. In some aspects, PBMCs comprise an immune cell. As used herein the term “immune cell” refers to any cell that plays a role in immune function. In some aspects, immune cell comprises a T cell, B cell, natural killer (NK) cell, dendritic cell (DC), NKT cell, mast cell, monocyte, macrophage, basophil, eosinophil, neutrophil, DC2.4 dendritic cell, or combinations thereof. Unless indicated otherwise, the term “PBMCs” comprises both bulk PBMCs and a subset thereof (e.g., population of T cells or monocytes isolated from the bulk PBMCs). In some aspects, the blood cells are red blood cells. In some aspects, the cell is a cancer cell. In some aspects, the cancer cell is a cancer cell line cell, such as a HeLa cell. In some aspects, the cancer cell is a tumor cell. In some aspects, the cancer cell is a circulating tumor cell (CTC). In some aspects, the cell is a fibroblast cell, such as a primary fibroblast or newborn human foreskin fibroblast (Nuff cell). In some aspects, the cell is an immortalized cell line cell, such as a HEK293 cell or a CHO cell. In some aspects, the cell is a skin cell. In some aspects, the cell is a reproductive cell such as an oocyte, ovum, or zygote. In some aspects, the cell is a cluster of cells, such as an embryo, given that the cluster of cells is not disrupted when passing through the pore.

In some aspects, the cell suspension useful for the present disclosure comprises a mixed or purified population of cells. In some aspects, the cell suspension is a mixed cell population (heterogeneous population of cells), such as whole blood, lymph, PBMCs, or combinations thereof. In some aspects, the cell suspension comprises bulk PBMCs which comprise a heterogeneous mixture of immune cells and myeloid cells (e.g., monocytes). In some aspects, the cell suspension is a purified cell population (homogeneous population of cells). For example, in some aspects, the cell suspension comprises a purified population of T cells. In some aspects, the cell suspension comprises a purified population of monocytes. Methods of obtaining such purified population of cells are known in the art (e.g., magnetic purification and/or flow cytometry-based sorting). In some aspects, the cell is a primary cell or a cell line cell.

As demonstrated herein, the delivery of a glial cell reprogramming factor into a cell can be regulated through one or more parameters of the process in which a cell suspension is passed through a constriction. In some aspects, the specific characteristics of the cell suspension can impact the delivery of a glial cell reprogramming factor into a cell. Such characteristics include, but are not limited to, osmolarity, salt concentration, serum content, cell concentration, pH, temperature, or combinations thereof. Additional parameters relevant for the present disclosure are provided elsewhere in the present disclosure.

In some aspects, the cell suspension comprises an aqueous solution. In some aspects, the aqueous solution comprises a cell culture medium, PBS, salts, sugars, growth factors, animal derived products, bulking materials, surfactants, lubricants, vitamins, polypeptides, an agent that impacts actin polymerization, or combinations thereof. In some aspects, the cell culture medium comprises DMEM, OptiMEM, EVIDM, RPMI, or combinations thereof. Additionally, solution buffer can include one or more lubricants (pluronics or other surfactants) that can be designed to reduce or eliminate clogging of the surface and improve cell viability. Exemplary surfactants include, without limitation, poloxamer, polysorbates, sugars such as mannitol, animal derived serum, and albumin protein.

When the suspension includes certain types of cells, in some aspects, the cells can be treated with a solution that aids in the delivery of the glial cell reprogramming factor (e.g., oligodendrocyte progenitor cell reprogramming factor) to the interior of the cell, such that the cell can be reprogrammed into glial cells. In some aspects, the solution comprises an agent that impacts actin polymerization. In some aspects, the agent that impacts actin polymerization comprises Latrunculin A, Cytochalasin, Colchicine, or combinations thereof. For example, in some aspects, the cells can be incubated in a depolymerization solution, such as Lantrunculin A, for about 1 hour prior to passing the cells through a constriction to depolymerize the actin cytoskeleton. In some aspects, the cells can be incubated in Colchicine (Sigma) for about 2 hours prior to passing the cells through a constriction to depolymerize the microtubule network.

In some aspects, a characteristic of a cell suspension that can affect the delivery of a glial cell reprogramming factor into a cell is the viscosity of the cell suspension. As used herein, the term “viscosity” refers to the internal resistance to flow exhibited by a fluid. In some aspects, the viscosity of the cell suspension is between about 8.9×10⁻⁴ Pa·s to about 4.0×10⁻³ Pa·s, between about 8.9×10⁻⁴ Pa·s to about 3.0×10⁻³ Pa·s, between about 8.9×10⁻⁴ Pa·s to about 2.0×10⁻³ Pa·s, or between about 8.9×10⁻⁴ Pa·s to about 1.0×10⁻³ Pa·s. In some aspects, the viscosity is between about 0.89 cP to about 4.0 cP, between about 0.89 cP to about 3.0 cP, between about 0.89 cP to about 2.0 cP, or between about 0.89 cP to about 1.0 cP. In some aspects, a shear thinning effect is observed, in which the viscosity of the cell suspension decreases under conditions of shear strain. Viscosity can be measured by any suitable method known in the art, including without limitation, viscometers, such as a glass capillary viscometer or rheometers. A viscometer measures viscosity under one flow condition, while a rheometer is used to measure viscosities which vary with flow conditions. In some aspects, the viscosity is measured for a shear thinning solution such as blood. In some aspects, the viscosity is measured between about 0° C. and about 45° C. For example, the viscosity of the cell suspension can be measured at room temperature (e.g., about 20° C.), physiological temperature (e.g., about 37° C.), higher than physiological temperature (e.g., greater than about 37° C. to about 45° C. or more), reduced temperature (e.g., about 0° C. to about 4° C.), or temperatures between these exemplary temperatures.

As described herein, the methods provided herein generally comprise contacting the cell suspension with a glial cell reprogramming factor through squeeze processing. In some aspects, the cell suspension can be further contacted with one or more additional payloads. Non-limiting examples of such additional compounds are provided elsewhere in the present disclosure. In some aspects, after a cell suspension passes through a constriction, the cell suspension can be contacted with a signaling molecule that is capable of inducing the formation of glial cells. Accordingly, in some aspects, the methods of producing glial cells (e.g., oligodendrocyte progenitor cells) provided herein comprises contacting the cell suspension with the glial cell reprogramming factor (e.g., prior to, during, and/or soon after the squeeze processing) and the signaling molecule. In some aspects, to contact the cell suspension with the signaling molecule, the cell suspension is collected after the squeeze processing, and then the cell suspension and the signaling molecule are cultured together. In some aspects, the cell suspension and the signaling molecules are cultured together for at least about one day, at least about two days, at least about three days, at least about four days, at least about five days, at least about six days, at least about seven days, at least about eight days, at least about nine days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, or at least about 30 days. In some aspects, the cell suspension and the signaling molecule are cultured together between about three days to about 21 days.

Non-limiting examples of signaling molecules that are useful for the present disclosure include: sonic hedgehog (SHH), basic fibroblast growth factor (bFGF), platelet-derived growth factor (PDGF), cAMP pathway activator (e.g., forskolin), TGF-β blocker (e.g., SB431542), BMP blocker (dorsomorphin), retinoic acid, rock-inhibitor (e.g., Y26732), insulin growth factor 1, ascorbic acid, neurotropic factors, or combinations thereof. In some aspects, neurotrophic factors comprise brain-derived neurotrophic factor (BDNF), glia-derived neurotrophic factor (GDNF), nerve growth factor (NGF), and cerebral dopamine neurotrophic factor (CDNF), or combinations thereof.

### III.B. Payloads

As described herein, in some aspects, a cell suspension additionally comprises one or more glial cell reprogramming factors (e.g., oligodendrocyte progenitor cell reprogramming factors) described herein. In some aspects, such a cell suspension can comprise one or more additional payloads that could be useful in combination with the glial cell reprogramming factors (e.g., payloads that help improve one or more properties of the glial cells).

As is apparent from the present disclosure, glial cell reprogramming factors useful for the present disclosure comprise any suitable payloads known in the art that can be delivered to a cell using the methods described herein and thereby, reprogram the cell to become a glial cell (e.g., OPC). Non-limiting examples of suitable payloads include a nucleic acid, a polypeptide, a lipid, a carbohydrate, a small molecule, a metal-containing compound, an antibody, a transcription factor, a nanoparticle, a liposome, a fluorescently tagged molecule, or combinations thereof. In some aspects, the nucleic acid comprises a DNA, RNA, or both. In some aspects, DNA comprises a recombinant DNA, a cDNA, a genomic DNA, or combinations thereof. In some aspects, RNA comprises a siRNA, a mRNA, a miRNA, a IncRNA, a tRNA, a shRNA, a self-amplifying mRNA (saRNA), a circular RNA, or combinations thereof. In some aspects, the RNA is mRNA. In some aspects, the RNA is siRNA. In some aspects, the RNA is shRNA. In some aspects, the RNA is miRNA. In some aspects, the RNA is a saRNA. In some aspects, the RNA is a circular RNA. In some aspects, a small molecule comprises an impermeable small molecule. As used herein, an “impermeable small molecule” refers to a small molecule that naturally does not cross the cell membrane of a cell

In some aspects, the glial cell reprogramming factor comprises an oligodendrocyte differentiation factor. As used herein, the term “glial cell differentiation factor” refers to any agent that is capable of inducing the differentiation of a cell (e.g., PBMCs, e.g., T cells or monocytes) into a glial cell (e.g., an oligodendrocyte progenitor cell). Accordingly, as used herein, the terms “glial cell reprogramming factor” and “glial cell differentiation factor” can be used interchangeably and refer to an agent that is capable of reprogramming and/or inducing a cell to become a glial cell. As used herein, a glial cell reprogramming factor is not particularly limited, as long as the agent is capable of inducing a cell (e.g., stem cell or PBMCs, e.g., T cells or monocytes) to differentiate into a glial cell (e.g., OPCs). Such glial cell reprogramming factors are known in the art. Non-limiting examples include: Yamanaka factors (OKSM) (e.g., Oct3/4, Sox2, c-Myc, Klf4), Oligodendrocyte Transcription Factor 2 (OLIG2), OLIG1, SRY-Box Transcription Factor 8 (SOX8), SOX9, SOX10, SOX5, SOX6, Myelin Regulatory Factor (MYRF), Myelin Transcription Factor 1 (MYT1), (NK2 Homeobox 2) NKX2.2, NKX6.1, NKX6.2, zinc finger protein 536 (ZFP536), (Achaete-scute homolog 1) ASCL1, ST18, NFIA, ZEB2, or combinations thereof. As is evident from the present disclosure, any of these glial cell reprogramming factors can be used alone or in combination.

In some aspects, the glial cell reprogramming factor is a Yamanaka factor. For instance, in some aspects, the glial cell reprogramming factor is OCT3/4. In some aspects, the glial cell reprogramming factor is SOX2. In some aspects, the glial cell reprogramming factor is C-MYC. In some aspects, the glial cell reprogramming factor is KLF4. In some aspects, the glial cell reprogramming factor is OLIG2. In some aspects, the glial cell reprogramming factor is OLIG1. In some aspects, the glial cell reprogramming factor is SOX8. In some aspects, the glial cell reprogramming factor is SOX9. In some aspects, the glial cell reprogramming factor is SOX10. In some aspects, the glial cell reprogramming factor is SOX5. In some aspects, the glial cell reprogramming factor is SOX6. In some aspects, the glial cell reprogramming factor is MYRF. In some aspects, the glial cell reprogramming factor is MYT1. In some aspects, the glial cell reprogramming factor is NKX2.2. In some aspects, the glial cell reprogramming factor is NKX6.1. In some aspects, the glial cell reprogramming factor is NKX6.2. In some aspects, the oligodendrocyte glial cell reprogramming factor is ZFP536. In some aspects, the glial cell reprogramming factor is ASCL1. In some aspects, the glial cell reprogramming factor is ST18. In some aspects, the glial cell reprogramming factor is NFIA. In some aspects, the glial cell reprogramming factor is ZEB2.

As further described and demonstrated herein, such glial cell reprogramming factors can be delivered to a cell (e.g., stem cells or PBMCs) alone or in combination (e.g., at least about two, at least about three, at least about four, at least about five, at least about six, at least about seven, at least about eight, at least about nine, at least about 10, at least about 11, or all of the exemplary reprogramming factors provided above). In some aspects, where combinations of glial cell reprogramming factors (or any other payloads described herein) are involved, they can be delivered to a cell using a single squeeze processing (e.g., a cell suspension comprises the multiple payloads, which are delivered to the cell in combination; “concurrent delivery”). In some aspects, the multiple glial cell reprogramming factors can be delivered to a cell sequentially. As used herein, the term “sequential delivery” refers to the delivery of multiple glial cell reprogramming factors to a cell, where a first glial cell reprogramming factor is delivered to the cell and then the second (or subsequent) glial cell reprogramming factor is delivered to the cell. In some aspects, the first glial cell reprogramming factor, the second glial cell reprogramming factor, or both the first and second glial cell reprogramming factors can be delivered to the cell using squeeze processing. For instance, in some aspects, the first glial cell reprogramming factor can be delivered to the cell using squeeze processing, and the second glial cell reprogramming factor can be delivered to the cell using non-squeeze processing (e.g., transfection). In some aspects, the first glial cell reprogramming factor can be delivered to the cell using non-squeeze processing (e.g., transfection), and the second glial cell reprogramming factor can be delivered to the cell using squeeze processing. In some aspects, the first glial cell reprogramming factor can be delivered to the cell using a first squeeze, and then the second glial cell reprogramming factor can be delivered to the cell using a second squeeze (also referred to herein as “sequential squeeze” or “sequential squeeze processing”). Accordingly, sequential delivery useful for the present disclosure can comprise multiple squeeze processings. In some aspects, each of the multiple squeeze processings delivers a separate glial cell reprogramming factor to the cell. In some aspects, one or more of the multiple squeeze processings do not involve the delivery of a glial cell reprogramming factor. For instance, in some aspects, a sequential delivery method described herein comprises a first squeeze, a second squeeze, and a third squeeze, wherein the first squeeze comprises passing a cell without any payload through a first constriction, the second squeeze comprises passing the cell from the first squeeze through a second constriction to deliver a first payload to the cell, and the third squeeze comprises passing the cell from the second squeeze through a third constriction to deliver a second payload to the cell. Not to be bound by any one theory, in some aspects, passing the cell through the first constriction without any payload (i.e., the first squeeze) can help prepare the cell for subsequent payload deliveries, e.g., can improve the delivery efficiency of the first payload and/or the second payload.

In some aspects, the glial cell reprogramming factors can be delivered to a cell (e.g., stem cells or PBMCs, e.g., T cells or monocytes) repeatedly (e.g., at least two times, at least three times, at least four times, at least five times or more) using the squeeze processing methods provided herein. For instance, in some aspects, a glial cell reprogramming factor (alone or in combination with other payloads) is delivered to cells with a first squeeze processing; then, the glial cell reprogramming factor (alone or in combination with other payloads) is delivered to the cells again with a second squeeze processing. In some aspects, the first squeeze processing includes a microfluidic device (e.g., chip) with multiple rows of constrictions, such that the squeeze process occurs on a single microfluidic device (e.g., chip). As further described herein, in some aspects, the second squeeze processing can occur immediately after the cells have gone through the first squeeze processing (e.g., immediately after the cells pass through the constriction of the first squeeze processing). In some aspects, the second squeeze processing can occur after some time after the first squeeze processing (e.g., at least about 1 minute, at least about 30 minutes, at least about 1 hour, at least about 6 hours, at least about 12 hours, or at least about 1 day after the cells pass through the constriction of the first squeeze processing). Accordingly, as further described elsewhere in the present disclosure (see, e.g., section titled “Constrictions”), in some aspects, each of the multiple squeeze processing methods can be the same (e.g., same parameters). In some aspects, one or more of the multiple squeeze processing methods can be different (e.g., one or more delivery parameters described herein are different).

Not to be bound by any one theory, in some aspects, a glial cell reprogramming factor useful for the present is capable of activating signaling pathways, such as those involved in oligodendrocyte differentiation. In some aspects, a glial cell reprogramming factor useful for the present disclosure is capable of inhibiting certain signaling pathways, such as those that interfere with oligodendrocyte differentiation.

In some aspects, the squeeze processing methods described herein can be used to deliver additional compounds, e.g., in combination with the payloads described above. As described elsewhere herein, the squeeze processing method of the present disclosure differs from the more traditional approaches to delivering payloads into cells to induce their reprogramming/differentiation. With the use of viral vectors (e.g., AAV or lentivirus) or with electroporation/lipofection, there are often cytotoxicity and/or homogeneity issues that make such approaches less desirable. With the present methods, as demonstrated herein, there are no lasting negative effects on the cells (e.g., majority of the squeeze-processed cells remain viable and resemble their non-squeeze-processed counterparts). Also, in some aspects, additional compounds can be delivered to cells using the present methods, wherein the additional compounds help improve one or more properties of a mixture comprising the reprogrammed/differentiated cells. In some aspects, the additional compound can comprise a nucleic acid, a polypeptide, a lipid, a carbohydrate, a small molecule, a metal-containing compound, an antibody, a transcription factor, a nanoparticle, a liposome, a fluorescently tagged molecule, or combinations thereof.

For instance, in some aspects, the additional compound is a nucleic acid encoding an enzyme that confers resistance to an antibiotic. Not to be bound by any one theory, by adding these compounds to the cell suspension comprising the plurality of cells and the glial cell reprogramming factor, it is possible to increase the purity of a mixture comprising the modified cells (i.e., reprogrammed/differentiated cells). By adding the particular antibiotic associated with the additional compound, the purity of a mixture comprising the modified cells (e.g., comprising OPCs) can be increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold. Non-limiting example such an additional compound includes puromycin-N-acetyltransferase and the antibiotic is puromycin.

### III.C. Constrictions

### III.C.1. Microfluidic Channels

As described herein, a constriction is used to cause a physical deformity in the cells, such that perturbations are created within the cell membrane of the cells, allowing for the delivery of a glial cell reprogramming factor into the cell. In some aspects, a constriction is within a channel contained within a microfluidic device (referred to herein as “microfluidic channel” or “channel”). Where multiple channels are involved, in some aspects, the multiple channels can be placed in parallel and/or in series within the microfluidic device. In some aspects, the cells described herein can be passed through at least about 2, at least about 3, at least about 4, at least about 5, at least about 6, at least about 7, at least about 8, at least about 9, at least about 10, at least about 20, at least about 30, at least about 40, at least about 50, at least about 75, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1,000 or more separate constrictions. In some aspects, the cells described herein are passed through more than about 1,000 separate constrictions.

In some aspects, the multiple constrictions can be part of a single microfluidic device (e.g., multi-row constriction chip). In some aspects, one or more of the multiple constrictions can be part of different microfluidic devices. For instance, in some aspects, the cells described herein (e.g., stem cells or PBMCs) undergo a first squeeze processing, in which the cells pass through a first constriction in a first microfluidic device (e.g., chip). Then, after the cells have undergone the first squeeze processing (e.g., passed through the first constriction), the cells undergo a second squeeze processing, in which the cells pass through a second constriction in a second microfluidic device (e.g., chip). In some aspects, each of the constrictions are the same (e.g., has the same length, width, and/or depth). In some aspects, one or more of the constrictions are different. Where plurality of constrictions are used, the plurality of constrictions can comprise a first constriction which is associated with a first glial cell reprogramming factor, and a second constriction which is associated with a second glial cell reprogramming factor, wherein the cell suspension passes through the first constriction such that the first glial cell reprogramming factor is delivered to one or more cells of the plurality of cells, and then the cell suspension passes through the second constriction such that the second glial cell reprogramming factor is delivered to the one or more cells of the plurality of cells. In some aspects, the cell suspension is passed through the second constriction at least about 1 minute, at least about 30 minutes, at least about 1 hour, at least about 6 hours, at least about 12 hours, or at least about 1 day after the cell suspension is passed through the first constriction.

In some aspects, where the cell suspension is passed through multiple constrictions (e.g., multiple squeeze processing), the cells remain viable after passing through each constriction. As is apparent from the present disclosure, in some aspects, multiple constrictions can comprise two or more constrictions present within a single microfluidic device (e.g., multi-row constriction chip), such the cells pass through the multiple constrictions sequentially. In some aspects, the multiple constrictions are part of separate microfluidic devices, such that a first constriction is associated with a first microfluidic device and a second constriction is associated with a second microfluidic device. For instance, in some aspects, cells are passed through a first constriction (i.e., first squeeze processing), which is associated with a first microfluidic device (e.g., chip). After the cells have passed through the first constriction, the cells are passed through a second constriction (i.e., second squeeze processing), which is associated with a second microfluidic device (e.g., chip). In some aspects, after passing through the first constriction, the cells are cultured in a medium prior to passing the cells through the second constriction. In some aspects, the cells are cultured for at least about 1 minute, at least about 30 minutes, at least about 1 hour, at least about 6 hours, at least about 12 hours, or at least about 1 day before passing the cells through the second constriction. As is apparent from the present disclosure, in some aspects, the first and second constrictions have the same length, depth, and/or width. In some aspects, the first and second constrictions can have different length, depth, and/or width.

In some aspects, after passing through a constriction, at least about 50%, at least about 55%, at least about 60%, at least about 65%, at least about 70%, at least about 75%, at least about 80%, at least about 85%, at least about 90%, at least about 95%, or about 100% of the cells remain viable. Where the cells pass through multiple constrictions (e.g., part of a single microfluidic device or separate microfluidic devices), at least about 50%, at least about 55%, at least about 60%, at least about 65%, at least about 70%, at least about 75%, at least about 80%, at least about 85%, at least about 90%, at least about 95%, or about 100% of the cells remain viable after passing through each of the multiple constrictions. The viability of the cells can be measured using any suitable methods known in the art. In some aspects, the viability of the cells can be measured using a Nucleocounter NC-200, an Orflo Moxi Go II Cell Counter, or both.

Exemplary microfluidic channels containing cell-deforming constrictions for use in the methods disclosed herein are described in US Publ. No. 2020/0277566 A1, US Publ. No. 2020/0332243 A1, US Publ. No. 2020/0316604 A1, U.S. Provisional Appl. No. 63/131,423, and U.S. Provisional Appl. No. 63/131,430, each of which is incorporated herein by reference in its entirety.

In some aspects, a microfluidic channel described herein (i.e., comprising a constriction) includes a lumen and is configured such that a cell suspended in a buffer (e.g., cell suspension) can pass through the channel. Microfluidic channels useful for the present disclosure can be made using any suitable materials available in the art, including, but not limited to, silicon, metal (e.g., stainless steel), plastic (e.g., polystyrene), ceramics, glass, crystalline substrates, amorphous substrates, polymers (e.g., Poly-methyl methacrylate (PMMA), PDMS, Cyclic Olefin Copolymer (COC)), or combinations thereof. In some aspects, the material is silicon. Fabrication of the microfluidic channel can be performed by any method known in the art, including, but not limited to, dry etching for example deep reactive ion etching, wet etching, photolithography, injection molding, laser ablation, SU-8 masks, or combinations thereof. In some aspects, the fabrication is performed using dry etching.

In some aspects, a microfluidic channel useful for the present disclosure comprises an entrance portion, a center point, and an exit portion. In some aspects, the cross-section of one or more of the entrance portion, the center point, and/or the exit portion can vary. For example, the cross-section can be circular, elliptical, an elongated slit, square, hexagonal, or triangular in shape.

The entrance portion defines a constriction angle. In some aspects, by modulating (e.g., increasing or decreasing) the constriction angle, any clogging of the constriction can be reduced or prevented. In some aspects, the angle of the exit portion can also be modulated. For example, in some aspects, the angle of the exit portion can be configured to reduce the likelihood of turbulence that can result in non-laminar flow. In some aspects, the walls of the entrance portion and/or the exit portion are linear. In some aspects, the walls of the entrance portion and/or the exit portion are curved.

In some aspects, the length, depth, and/or width of the constriction can vary. In some aspects, by modulating (e.g., increasing or decreasing) the length, depth, and/or width of the constriction, the delivery efficiency of a payload can be regulated. As used herein, the term “delivery efficiency” refers to the amount of payload that is delivered into the cell. For instance, an increased delivery efficiency can occur when the total amount of payload that is delivered is increased.

In some aspects, the constriction has a length of less than about 1 μm. In some aspects the constriction has a length of about 0.1 μm to about 100 μm. In some aspects, the constriction has a length of less than about 0.1 μm, about 0.2 μm, about 0.3 μm, about 0.4 μm, about 0.5 μm, about 0.6 μm, about 0.7 μm, about 0.8 μm, about 0.9 μm, about 1 μm, about 2.5 μm, about 5 μm, about 7.5 μm, about 10 μm, about 12.5 μm, about 15 μm, about 20 μm, about 30 μm, about 40 μm, about 50 μm, about 60 μm, about 70 μm, about 80 μm, about 90 μm, or about 100 μm. In some aspects, the constriction has a length of about 10 μm. In some aspects, the constriction has a length of about 70 μm.

In some aspects, the constriction has a depth of about 5 μm to about 90 μm. In some aspects, the constriction has a depth greater than or equal to about 5 μm, about 10 μm, about 20 μm, about 30 μm, about 40 μm, about 50 μm, about 60 μm, about 70 μm, about 80 μm, about 90 μm, about 100 μm, about 110 μm, or about 120 μm. In some aspects, the constriction has a depth of about 10 μm. In some aspects, the constriction has a depth of about 70 μm.

In some aspects, the constriction has a width of about 1 μm to about 10 μm. In some aspects, the constriction has a width of about 1 μm, about 1.1 μm, about 1.2 μm, about 1.3 μm, about 1.4 μm, about 1.5 μm, about 1.6 μm, about 1.7 μm, about 1.8 μm, about 1.9 μm, about 2 μm, about 3 μm, about 4 μm, about 4.5 μm, about 5 μm, about 6 μm, about 7 μm, about 8 μm, about 9 μm, or about 10 μm. In some aspects, the constriction has a width of about 6 μm. In some aspects, the constriction has a width of about 6 μm. In some aspects, the constriction has a length of 10 μm, width of 6 μm, and a depth of 70 μm. In some aspects, the constriction has a width of about 3.5 μm. For example, in some aspects, the constriction that can used with the present disclosure has a depth of 10 μm, width of 3.5 μm, and a length of 70 μm.

In some aspects, the diameter of a constriction (e.g., contained within a microfluidic channel) is a function of the diameter of one or more cells that are passed through the constriction. Not to be bound by any one theory, in some aspects, the diameter of the constriction is less than that of the cells, such that a deforming force is applied to the cells as they pass through the constriction, resulting in the transient physical deformity of the cells.

Accordingly, in some aspects, the diameter of the constriction (also referred to herein as “constriction size”) is about 20% to about 99% of the diameter of the cell. In some aspects, the constriction size is about 20%, about 30%, about 40%, about 50%, about 60%, about 70%, about 80%, about 90%, or about 99% of the cell diameter. As is apparent from the present disclosure, by modulating (e.g., increasing or decreasing) the diameter of a constriction, the delivery efficiency of a payload into a cell can also be regulated.

### III.C.2. Surfaces Having Pores

In some aspects, a constriction described herein comprises a pore, which is contained in a surface. Non-limiting examples of pores contained in a surface that can be used with the present disclosure are described in, e.g., US Publ. No. 2019/0382796 A1, which is incorporated herein by reference in its entirety.

In some aspects, a surface useful for the present disclosure (i.e., comprising one or more pores that can cause a physical deformity in a cell as it passes through the pore) can be made using any suitable materials available in the art and/or take any one of a number of forms. Non-limiting examples of such materials include synthetic or natural polymers, polycarbonate, silicon, glass, metal, alloy, cellulose nitrate, silver, cellulose acetate, nylon, polyester, polyethersulfone, polyacrylonitrile (PAN), polypropylene, PVDF, polytetrafluorethylene, mixed cellulose ester, porcelain, ceramic, or combinations thereof.

In some aspects, the surface comprises a filter. In some aspects, the filter is a tangential flow filter. In some aspects, the surface comprises a membrane. In some aspects, the surface comprises a sponge or sponge-like matrix. In some aspects, the surface comprises a matrix. In some aspects, the surface comprises a tortuous path surface. In some aspects, the tortuous path surface comprises cellulose acetate.

The surface disclosed herein (i.e., comprising one or more pores) can have any suitable shape known in the art. Where the surface has a 2-dimensional shape, the surface can be, without limitation, circular, elliptical, round, square, star-shaped, triangular, polygonal, pentagonal, hexagonal, heptagonal, or octagonal. In some aspects, the surface is round in shape. Where the surface has a 3-dimensional shape, in some aspects, the surface can be, without limitation, cylindrical, conical, or cuboidal.

As is apparent from the present disclosure, a surface that is useful for the present disclosure (e.g., comprising one or more pores) can have various cross-sectional widths and thicknesses. In some aspects, the cross-sectional width of the surface is between about 1 mm and about 1 m. In some aspects, the surface has a defined thickness. In some aspects, the surface thickness is uniform. In some aspects, the surface thickness is variable. For example, in some aspects, certain portions of the surface are thicker or thinner than other portions of the surface. In such aspects, the thickness of the different portions of the surface can vary by about 1% to about 90%. In some aspects, the surface is between about 0.01 μm to about 5 mm in thickness.

The cross-sectional width of the pores can depend on the type of cell that is being targeted with a payload. In some aspects, the pore size is a function of the diameter of the cell of cluster of cells to be targeted. In some aspects, the pore size is such that a cell is perturbed (i.e., physically deformed) upon passing through the pore. In some aspects, the pore size is less than the diameter of the cell. In some aspects, the pore size is about 20% to about 99% of the diameter of the cell. In some aspects, the pore size is about 20%, about 30%, about 40%, about 50%, about 60%, about 70%, about 80%, about 90%, or about 99% of the diameter of the cell. In some aspects, the pore size is about 0.4 μm, about 0.5 μm, about 0.6 μm, about 0.7 μm, about 0.8 μm, about 0.9 μm, about 1 μm, about 2 μm, about 3 μm, about 4 μm, about 5 μm, about 6 μm, about 7 μm, about μm, about 9 μm, about 10 μm, about 11 μm, about 12 μm, about 13 μm, about 14 μm, or about 15 μm or more.

The entrances and exits of a pore can have a variety of angles. In some aspects, by modulating (e.g., increasing or decreasing) the pore angle, any clogging of the pore can be reduced or prevented. In some aspects, the flow rate (i.e., the rate at which a cell or a suspension comprising the cell passes through the pore) is between about 0.001 mL/cm/sec to about 100 L/cm/sec. For example, the angle of the entrance or exit portion can be between about 0 and about 90 degrees. In some aspects, the pores have identical entrance and exit angles. In some aspects, the pores have different entrance and exit angles. In some aspects, the pore edge is smooth, e.g., rounded or curved. As used herein, a “smooth” pore edge has a continuous, flat, and even surface without bumps, ridges, or uneven parts. In some aspects, the pore edge is sharp. As used herein, a “sharp” pore edge has a thin edge that is pointed or at an acute angle. In some aspects, the pore passage is straight. As used herein, a “straight” pore passage does not contain curves, bends, angles, or other irregularities. In some aspects, the pore passage is curved. As used herein, a “curved” pore passage is bent or deviates from a straight line. In some aspects, the pore passage has multiple curves, e.g., about 2, about 3, about 4, about 5, about 6, about 7, about 8, about 9, about 10 or more curves.

The pores can have any shape known in the art, including a 2-dimensional or 3-dimensional shape. The pore shape (e.g., the cross-sectional shape) can be, without limitation, circular, elliptical, round, square, star-shaped, triangular, polygonal, pentagonal, hexagonal, heptagonal, and octagonal. In some aspects, the cross-section of the pore is round in shape. In some aspects, the 3-dimensional shape of the pore is cylindrical or conical. In some aspects, the pore has a fluted entrance and exit shape. In some aspects, the pore shape is homogenous (i.e., consistent or regular) among pores within a given surface. In some aspects, the pore shape is heterogeneous (i.e., mixed or varied) among pores within a given surface.

A surface useful for the present disclosure can have a single pore. In some aspects, a surface useful for the present disclosure comprises multiple pores. In some aspects, the pores encompass about 10% to about 80% of the total surface area of the surface. In some aspects, the surface contains about 1.0×10⁵ to about 1.0×10³⁰ total pores. In some aspects, the surface comprises between about 10 and about 1.0×10¹⁵ pores per mm² surface area.

The pores can be distributed in numerous ways within a given surface. In some aspects, the pores are distributed in parallel within a given surface. In some aspects, the pores are distributed side-by-side in the same direction and are the same distance apart within a given surface. In some aspects, the distribution of the pores is ordered or homogeneous. In such aspects, the pores can be distributed in a regular, systematic pattern, or can be the same distance apart within a given surface. In some aspects, the distribution of the pores is random or heterogeneous. For instance, in some aspects, the pores are distributed in an irregular, disordered pattern, or are different distances apart within a given surface.

In some aspects, multiple surfaces are used, such that a cell passes through multiple pores, wherein the pores are on different surfaces. In some aspects, multiple surfaces are distributed in series. The multiple surfaces can be homogeneous or heterogeneous in surface size, shape, and/or roughness. The multiple surfaces can further contain pores with homogeneous or heterogeneous pore size, shape, and/or number, thereby enabling the simultaneous delivery of a range of payloads into different cell types.

In some aspects, an individual pore, e.g., of a surface that can be used with the present disclosure, has a uniform width dimension (i.e., constant width along the length of the pore passage). In some aspects, an individual pore has a variable width (i.e., increasing or decreasing width along the length of the pore passage). In some aspects, pores within a given surface have the same individual pore depths. In some aspects, pores within a given surface have different individual pore depths. In some aspects, the pores are immediately adjacent to each other. In some aspects, the pores are separated from each other by a distance. In some aspects, the pores are separated from each other by a distance of about 0.001 μm to about 30 mm.

In some aspects, the surface is coated with a material. The material can be selected from any material known in the art, including, without limitation, Teflon, an adhesive coating, surfactants, proteins, adhesion molecules, antibodies, anticoagulants, factors that modulate cellular function, nucleic acids, lipids, carbohydrates, transmembrane proteins, or combinations thereof. In some aspects, the surface is coated with polyvinylpyrrolidone. In some aspects, the material is covalently attached to the surface. In some aspects, the material is non-covalently attached to the surface. In some aspects, the surface molecules are released at the cells pass through the pores.

In some aspects, the surface has modified chemical properties. In some aspects, the surface is hydrophilic. In some aspects, the surface is hydrophobic. In some aspects, the surface is charged. In some aspects, the surface is positively and/or negatively charged. In some aspects, the surface can be positively charged in some regions and negatively charged in other regions. In some aspects, the surface has an overall positive or overall negative charge. In some aspects, the surface can be any one of smooth, electropolished, rough, or plasma treated. In some aspects, the surface comprises a zwitterion or dipolar compound. In some aspects, the surface is plasma treated.

In some aspects, the surface is contained within a larger module. In some aspects, the surface is contained within a syringe, such as a plastic or glass syringe. In some aspects, the surface is contained within a plastic filter holder. In some aspects, the surface is contained within a pipette tip.

### III.D. Cell Perturbation

As described herein, as a cell passes through a constriction, it becomes physically deformed, such that there is a perturbation (e.g., a hole, tear, cavity, aperture, pore, break, gap, perforation) in the cell membrane of the cell. Such perturbation in the cell membrane is temporary and sufficient for any of the glial cell reprogramming factors described herein to be delivered into the cell. Cells have self-repair mechanisms that allow the cells to repair any disruption in their cell membrane. See Blazek et al., Physiology (Bethesda) 30(6): 438-48 (November 2015), which is incorporated herein by reference in its entirety. Accordingly, in some aspects, once the cells have passed through the constriction (e.g., microfluidic channel or pores), the perturbations in the cell membrane can be reduced or eliminated, such that the payload that was delivered into the cell does not exit the cell.

In some aspects, the perturbation in the cell membrane lasts from about 1.0×10⁻⁹ seconds to about 2 hours after the pressure is removed (e.g., cells have passed through the constriction). In some aspects, the cell perturbation lasts for about 1.0×10⁻⁹ second to about 1 second, for about 1 second to about 1 minute, or for about 1 minute to about 1 hour. In some aspects, the cell perturbation lasts for between about 1.0×10⁻⁹ second to about 1.0×10⁻¹ second, between about 1.0×10⁻⁹ second to about 1.0×10⁻² second, between about 1.0×10⁻⁹ second to about 1.0×10⁻³ second, between about 1.0×10⁻⁹ second to about 1.0×10⁻⁴ second, between about 1.0×10⁻⁹ second to about 1.0×10⁻⁵ second, between about 1.0×10⁻⁹ second to about 1.0×10⁻⁶ second, between about 1.0×10⁻⁹ second to about 1.0×10⁻⁷ second, or between about 1.0×10⁻⁹ second to about 1.0×10⁻⁸ second. In some aspects, the cell perturbation lasts for about 1.0×10⁻⁸ second to about 1.0×10⁻¹ second, for about 1.0×10⁻⁷ second to about 1.0×10⁻¹ second, about 1.0×10⁻⁶ second to about 1.0×10⁻¹ second, about 1.0×10⁻⁵ second to about 1.0×10⁻¹ second, about 1.0×10⁻⁴ second to about 1.0×10⁻¹ second, about 1.0×10⁻³ second to about 1.0×10⁻¹ second, or about 1.0×10⁻² second to about 1.0×10⁻¹ second. The cell perturbations (e.g., pores or holes) created by the methods described herein are not formed as a result of assembly of polypeptide subunits to form a multimeric pore structure such as that created by complement or bacterial hemolysins.

In some aspects, as the cell passes through the constriction, the pressure applied to the cells temporarily imparts injury to the cell membrane that causes passive diffusion of material through the perturbation. In some aspects, the cell is only deformed or perturbed for a brief period of time, e.g., on the order of 100 μs or less to minimize the chance of activating apoptotic pathways through cell signaling mechanisms, although other durations are possible (e.g., ranging from nanoseconds to hours). In some aspects, the cell is deformed for less than about 1.0×10⁻⁹ second to less than about 2 hours. In some aspects, the cell is deformed for less than about 1.0×10⁻⁹ second to less than about 1 second, less than about 1 second to less than about 1 minute, or less than about 1 minute to less than about 1 hour. In some aspects, the cell is deformed for about 1.0×10⁻⁹ second to about 2 hours. In some aspects, the cell is deformed for about 1.0×10⁻⁹ second to about 1 second, about 1 second to about 1 minute, or about 1 minute to about 1 hour. In some aspects, the cell is deformed for between any one of about 1.0×10⁻⁹ second to about 1.0×10⁻¹ second, about 1.0×10⁻⁹ second to about 1.0×10⁻² second, about 1.0×10⁻⁹ second to about 1.0×10⁻³ second, about 1.0×10⁻⁹ second to about 1.0×10⁻⁴ second, about 1.0×10⁻⁹ second to about 1.0×10⁻⁵ second, about 1.0×10⁻⁹ second to about 1.0×10⁻⁶ second, about 1.0×10⁻⁹ second to about 1.0×10⁻⁷ second, or about 1.0×10⁻⁹ second to about 1.0×10⁻⁸ second. In some aspects, the cell is deformed or perturbed for about 1.0×10⁻⁸ second to about 1.0×10⁻¹ second, for about 1.0×10⁻⁷ second to about 1.0×10⁻¹ second, about 1.0×10⁻⁶ second to about 1.0×10⁻¹ second, about 1.0×10⁻⁵ second to about 1.0×10⁻¹ second, about 1.0×10⁻⁴ second to about 1.0×10⁻¹ second, about 1.0×10⁻³ second to about 1.0×10⁻¹ second, or about 1.0×10⁻² second to about 1.0×10⁻¹ second. In some aspects, deforming the cell includes deforming the cell for a time ranging from, without limitation, about 1 μs to at least about 750 μs, e.g., at least about 1 μs, at least about 10 μs, at least about 50 μs, at least about 100 μs, at least about 500 μs, or at least about 750 μs.

In some aspects, the delivery of a glial cell reprogramming factor into the cell occurs simultaneously with the cell passing through the constriction. In some aspects, delivery of the glial cell reprogramming factor into the cell can occur after the cell passes through the constriction (i.e., when perturbation of the cell membrane is still present and prior to cell membrane of the cells being restored). In some aspects, delivery of the glial cell reprogramming factor into the cell occurs on the order of minutes after the cell passes through the constriction. In some aspects, a perturbation in the cell after it passes through the constriction is corrected within the order of about five minutes after the cell passes through the constriction.

In some aspects, the viability of a cell (e.g., stem cell or PBMC) after passing through a constriction is about 5% to about 100%. In some aspects, the cell viability after passing through the constriction is at least about 5%, at least about 10%, at least about 20%, at least about 30%, at least about 40%, at least about 50%, at least about 60%, at least about 70%, at least about 75%, at least about 80%, at least about 85%, at least about 90%, at least about 95%, or at least about 99%. In some aspects, the cell viability is measured from about 1.0×10⁻² second to at least about 10 days after the cell passes through the constriction. For example, the cell viability can be measured from about 1.0×10⁻² second to about 1 second, about 1 second to about 1 minute, about 1 minute to about 30 minutes, or about 30 minutes to about 2 hours after the cell passes through the constriction. In some aspects, the cell viability is measured about 1.0×10⁻² second to about 2 hours, about 1.0×10⁻² second to about 1 hour, about 1.0×10⁻² second to about 30 minutes, about 11.0×10⁻² second to about 1 minute, about 1.0×10⁻² second to about 30 seconds, about 1.0×10⁻² second to about 1 second, or about 1.0×10⁻² second to about 0.1 second after the cell passes through the constriction. In some aspects, the cell viability is measured about 1.5 hours to about 2 hours, about 1 hour to about 2 hours, about 30 minutes to about 2 hours, about 15 minutes to about 2 hours, about 1 minute to about 2 hours, about 30 seconds to about 2 hours, or about 1 second to about 2 hours after the cell passes through the constriction. In some aspects, the cell viability is measured about 2 hours to about 5 hours, about 5 hours to about 12 hours, about 12 hours to about 24 hours, or about 24 hours to about 10 days after the cell passes through the constriction.

### III.E. Delivery Parameters

As is apparent from the present disclosure, a number of parameters can influence the delivery efficiency of a glial cell reprogramming factor into a cell using the squeeze processing methods provided herein. Accordingly, by modulating (e.g., increasing or decreasing) one or more of the delivery parameters, the delivery of the glial cell reprogramming factor into a cell can be improved. Therefore, in some aspects, the present disclosure relates to a method of increasing the delivery of a glial cell reprogramming factor into a cell, wherein the method comprises modulating one or more parameters under which a cell suspension is passed through a constriction, wherein the cell suspension comprises a population of the cells, and wherein the one or more parameters increase the delivery of the glial cell reprogramming factor into one or more cells of the population of cells compared to a reference parameter. As described elsewhere in the present disclosure, the glial cell reprogramming factor can be in contact with the population of cells before, during, and/or after the squeezing step.

In some aspects, by modulating one or more of the delivery parameters, the delivery of the glial cell reprogramming factor into the one or more cells is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, compared to a delivery of the glial cell reprogramming factor into a corresponding cell using the reference parameter.

In some aspects, the one or more delivery parameters that can be modulated to increase the delivery efficiency of a parameter comprises a cell density (i.e., the concentration of the cells present, e.g., in the cell suspension), pressure, or both. Additional examples of delivery parameters that can be modulated are provided elsewhere in the present disclosure.

In some aspects, the cell density is about 1×10⁵ cells/mL, about 2×10⁵ cells/mL, about 3×10⁵ cells/mL, about 4×10⁵ cells/mL, about 5×10⁵ cells/mL, about 6×10⁵ cells/mL, about 7×10⁵ cells/mL, about 8×10⁵ cells/mL, about 9×10⁵ cells/mL, about 1×10⁶ cells/mL, about 2×10⁶ cells/mL, about 3×10⁶ cells/mL, about 4×10⁶ cells/mL, about 5×10⁶ cells/mL, about 6×10⁶ cells/mL, about 7×10⁶ cells/mL, about 8×10⁶ cells/mL, about 9×10⁶ cells/mL, about 1×10⁷ cells/mL, about 2×10⁷ cells/mL, about 3×10⁷ cells/mL, about 4×10⁷ cells/mL, about 5×10⁷ cells/mL, about 6×10⁷ cells/mL, about 7×10⁷ cells/mL, about 8×10⁷ cells/mL, about 9×10⁷ cells/mL, about 1×10⁸ cells/mL, about 1.1×10⁸ cells/mL, about 1.2×10⁸ cells/mL, about 1.3×10⁸ cells/mL, about 1.4×10⁸ cells/mL, about 1.5×10⁸ cells/mL, about 2.0×10⁸ cells/mL, about 3.0×10⁸ cells/mL, about 4.0×10⁸ cells/mL, about 5.0×10⁸ cells/mL, about 6.0×10⁸ cells/mL, about 7.0×10⁸ cells/mL, about 8.0×10⁸ cells/mL, about 9.0×10⁸ cells/mL, or about 1.0×10⁹ cells/mL or more. In some aspects, the cell density is between about 6×10⁷ cells/mL and about 1.2×10⁸ cells/mL.

In some aspects, the pressure is about 20 psi, about 25 psi, about 30 psi, about 35 psi, about 40 psi, about 50 psi, about 55 psi, about 60 psi, about 65 psi, about 70 psi, about 75 psi, about 80 psi, about 85 psi, about 90 psi, about 95 psi, about 100 psi, about 110 psi, about 120 psi, about 130 psi, about 140 psi, about 150 psi, about 160 psi, about 170 psi, about 180 psi, about 190 psi, or about 200 psi or more. In some aspects, the pressure is between about 30 psi and about 90 psi. In some aspects, the pressure is about 60 psi.

In some aspects, the particular type of device (e.g., microfluidic chip) can also have an effect on the delivery efficiency of a glial cell reprogramming factor. In the case of a microfluidic chip, different chips can have different constriction parameters, e.g., length, depth, and width of the constriction; entrance angle, exit angle, length, depth, and width of the approach region, etc. As described herein, such variables can influence the delivery of a glial cell reprogramming factor into a cell using the squeeze processing methods of the present disclosure.

In some aspects, the length of the constriction is up to about 100 μm. For instance, in some aspects, the length is about 0 μm, about 0.1 μm, about 0.2 μm, about 0.3 μm, about 0.4 μm, about 0.5 μm, about 0.6 μm, about 0.7 μm, about 0.8 μm, about 0.9 μm, about 1 μm, about 2.5 μm, about 5 μm, about 7.5 μm, 10 μm, about 12.5 μm, about 15 μm, about 20 μm, about 30 μm, about 40 μm, about 50 μm, about 60 μm, about 70 μm, about 80 μm, about 90 μm, or about 100 μm. In some aspects, the length of the constriction is less than about 1 μm. In some aspects, the length of the constriction is less than about 0.1 μm, less than about 0.2 μm, less than about 0.3 μm, less than about 0.4 μm, less than about 0.5 μm, less than about 0.6 μm, less than about 0.7 μm, less than about 0.8 μm, less than about 0.9 μm, less than about 1 μm, less than about 2.5 μm, less than about 5 μm, less than about 7.5 μm, less than about 10 μm, less than about 12.5 μm, less than about 15 μm, less than about 20 μm, less than about 30 μm, less than about 40 μm, less than about 50 μm, less than about 60 μm, less than about 70 μm, less than about 80 μm, less than about 90 μm, or less than about 100 μm. In some aspects, the constriction has a length of about 10 μm. In some aspects, the constriction has a length of about 0.5 μm. In some aspects, the constriction has a length of about 70 μm. In some aspects, the constriction has a length of about 0 μm. For example, in some aspects, a microfluidic device (e.g., chip) useful for the present disclosure comprises a constriction that resembles two points of a diamond coming together, such that the length of the constriction is about 0 μm.

In some aspects, the width of the constriction is up to about 10 μm. In some aspects, the width of the constriction is less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm. In some aspects, the width is between about 1 μm to about 10 μm (e.g., 3 μm to about 10 μm). In some aspects, the width is about 1 μm, about 1.1 μm, about 1.2 μm, about 1.3 μm, about 1.4 μm, about 1.5 μm, about 1.6 μm, about 1.7 μm, about 1.8 μm, about 1.9 μm, about 2.0 μm, about 3 μm, about 4 μm, about 5 μm, about 6 μm, about 7 μm, about 8 μm, about 9 μm, or about 10 μm. In some aspects, the width of the constriction is about 6 μm. In some aspects, the constriction has a width of about 3.5 μm.

In some aspects, the depth of the constriction is at least about 1 μm. In some aspects, the depth of the constriction is at least about 1 μm, at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm. In some aspects, the depth is between about 5 μm to about 90 μm. In some aspects, the depth is about 5 μm, about 10 μm, about 15 μm, about 20 μm, about 30 μm, about 40 μm, about 50 μm, about 60 μm, about 70 μm, about 80 μm, or about 90 μm. In some aspects, the depth of the constriction is about 70 μm. In some aspects, the constriction has a depth of about 10 μm.

In some aspects, the length is about 10 μm, the width is about 6 μm, and depth is about 70 μm, and the pressure is about 60 psi. In some aspects, the depth is about 10 μm, the width is about 3.5 μm, and length is about 70 μm, and the pressure is about 20 psi.

Additional examples of parameters that can influence the delivery of a glial cell reprogramming factor into the cell include, but are not limited to, the dimensions of the constriction (e.g., length, width, and/or depth), the entrance angle of the constriction, the surface properties of the constrictions (e.g., roughness, chemical modification, hydrophilic, hydrophobic), the operating flow speeds, glial cell reprogramming factor concentration, the amount of time that the cell recovers, or combinations thereof. Further parameters that can influence the delivery efficiency of a glial cell reprogramming factor can include the velocity of the cell in the constriction, the shear rate in the constriction, the viscosity of the cell suspension, the velocity component that is perpendicular to flow velocity, and time in the constriction. Such parameters can be designed to control delivery of the glial cell reprogramming factor.

In some aspects, the temperature used in the methods of the present disclosure can also have an effect on the delivery efficiency of the glial cell reprogramming factor into the cell, as well as the viability of the cell. In some aspects, the squeeze processing method is performed between about −5° C. and about 45° C. For example, the methods can be carried out at room temperature (e.g., about 20° C.), physiological temperature (e.g., about 37° C.), higher than physiological temperature (e.g., greater than about 37° C. to 45°° C. or more), or reduced temperature (e.g., about −5° C. to about 4° C.), or temperatures between these exemplary temperatures.

Various methods can be utilized to drive the cells through the constrictions. For example, pressure can be applied by a pump on the entrance side (e.g., gas cylinder, or compressor), a vacuum can be applied by a vacuum pump on the exit side, capillary action can be applied through a tube, and/or the system can be gravity fed. Displacement based flow systems can also be used (e.g., syringe pump, peristaltic pump, manual syringe or pipette, pistons, etc.). In some aspects, the cells are passed through the constrictions by positive pressure. In some aspects, the cells are passed through the constrictions by constant pressure or variable pressure. In some aspects, pressure is applied using a syringe. In some aspects, pressure is applied using a pump. In some aspects, the pump is a peristaltic pump or a diaphragm pump. In some aspects, pressure is applied using a vacuum. In some aspects, the cells are passed through the constrictions by g-force. In some aspects, the cells are passed through the constrictions by capillary pressure.

In some aspects, fluid flow directs the cells through the constrictions. In some aspects, the fluid flow is turbulent flow prior to the cells passing through the constriction. Turbulent flow is a fluid flow in which the velocity at a given point varies erratically in magnitude and direction. In some aspects, the fluid flow through the constriction is laminar flow. Laminar flow involves uninterrupted flow in a fluid near a solid boundary in which the direction of flow at every point remains constant. In some aspects, the fluid flow is turbulent flow after the cells pass through the constriction. The velocity at which the cells pass through the constrictions can be varied. In some aspects, the cells pass through the constrictions at a uniform cell speed. In some aspects, the cells pass through the constrictions at a fluctuating cell speed.

In some aspects, a combination treatment is used to deliver a glial cell reprogramming factor, e.g., the methods described herein followed by exposure to an electric field downstream of the constriction. In some aspects, the cell is passed through an electric field generated by at least one electrode after passing through the constriction. In some aspects, the electric field assists in delivery of a glial cell reprogramming factor to a second location inside the cell such as the cell nucleus. In some aspects, one or more electrodes are in proximity to the cell-deforming constriction to generate an electric field. In some aspects, the electric field is between about 0.1 kV/m to about 100 MV/m. In some aspects, an integrated circuit is used to provide an electrical signal to drive the electrodes. In some aspects, the cells are exposed to the electric field for a pulse width of between about 1 ns to about 1 s and a period of between about 100 ns to about 10 s.

### III.F. Therapeutic Uses

In some aspects, the present disclosure relates to the use of the cells (e.g., oligodendrocytes) produced using the squeeze processing methods described herein to treat various diseases or disorders. As is apparent from the present disclosure, the methods and compositions provided herein can be useful for diseases and disorders where cell replacement therapies can be used as a treatment. By replacing cells that are damaged with the cells produced using the methods provided herein, in some aspects, one or more functions associated with the damaged cells can be restored, and thereby, treat the disease or disorder. For instance, in some aspects, oligodendrocytes that are produced using the squeeze processing methods provided herein could be administered to a subject suffering from a demyelinating disease. The administration of such oligodendrocytes could be useful in improving one or more symptoms associated with the demyelinating disease (e.g., by inducing the remyelination of demyelinated neurons). Non-limiting examples of demyelinating diseases that can be treated with the present disclosure include: multiple sclerosis. As used herein, the term “multiple sclerosis” (MS) refers to a chronic and often disabling disease of the central nervous system characterized by the progressive destruction of the myelin sheath. Multiple sclerosis is generally diagnosed as one of four internationally recognized categories or stages of MS: (1) primary progressive multiple sclerosis (PPMS), (2) relapsing-remitting multiple sclerosis (RRMS), (3) secondary progressive multiple sclerosis (SPMS), and (4) progressive relapsing multiple sclerosis. Standards for diagnosis are known to those of skill in the art and exemplary diagnostic criteria are described in, e.g., “Merck Manual, Professional Version” (worldwideweb.merckmanuals.com/professional/neurologic-disorders/demyelinating-disorders/multiple-sclerosis-ms). Unless indicated otherwise, the term “multiple sclerosis” encompasses all of the different categories of MS and related disorders. For example, in some aspects, multiple sclerosis comprises “clinically isolated syndrome” (CIS). A CIS refers to an isolated (single) episode of neurologic symptoms (lasting at least about 24 hours) caused by inflammation and damage to myelin. The episode is characteristic of multiple sclerosis but fails to meet the diagnosis criteria of multiple sclerosis. The subject may or may not subsequently develop multiple sclerosis. Subjects with clinically isolated syndrome can present with lesions on the brain which increase the chances of the subject having a subsequent episode and developing multiple sclerosis. Accordingly, in some aspects, the present disclosure can be used to treat all types of MS, including a clinically isolated syndrome (CIS), relapsing-remitting MS (RRMS), secondary progressive MS (SPMS), primary progressive MS (PPMS), or a combination thereof.

### IV. Compositions of the Disclosures

In some aspects, the disclosure provides a system for delivery of a glial cell reprogramming factor into a cell, the system comprising a microfluidic channel described herein, a cell suspension comprising a plurality of the cells and the glial cell reprogramming factor; wherein the constriction is configured such that the plurality of cells can pass through the microfluidic channel, wherein the passing of the plurality of cells causes a deformity and disruption of the cell membrane of the cell, allowing the glial cell reprogramming factor to enter the cell.

In some aspects, the disclosure provides a system for delivering a glial cell reprogramming factor, the system comprising a surface with pores, a cell suspension comprising a plurality of the cells and the glial cell reprogramming factor; wherein the surface with pores is configured such that the plurality of cells can pass through the pores, wherein the passing of the plurality of cells causes a transient deformity and disruption of the cell membrane of the cell, allowing the glial cell reprogramming factor to enter the cell. In some aspects, the surface is a filter or a membrane. In some of the above aspects, the system further comprises at least one electrode to generate an electric field. In some aspects, the system is used to deliver a glial cell reprogramming factor into a cell by any of the methods described herein. The system can include any aspect described for the methods disclosed above, including microfluidic channels or a surface having pores to provide cell-deforming constrictions, cell suspensions, cell perturbations, delivery parameters. In some aspects, the delivery parameters, such as operating flow speeds, cell and compound concentration, velocity of the cell in the constriction, and the composition of the cell suspension (e.g., osmolarity, salt concentration, serum content, cell concentration, pH, etc.) are optimized for delivery of a payload (e.g., glial cell reprogramming factor) into the cell.

In some aspects, the disclosure provides a cell produced using any of the methods provided herein (e.g., OPCs that can further differentiate into oligodendrocytes). In some aspects, provided herein is a cell comprising a perturbation in the cell membrane, wherein the perturbation is due to one or more parameters which deform the cell (e.g., delivery parameters described herein), thereby creating the perturbation in the cell membrane of the cell such that a glial cell reprogramming factor can enter the cell. In some aspects, provided herein is a cell comprising a glial cell reprogramming factor, wherein the glial cell reprogramming factor entered the cell through a perturbation in the cell membrane, which was due to one or more parameters which deform the cell (e.g., delivery parameters described herein) and thereby creating the perturbation in the cell membrane of the cell such that the glial cell reprogramming factor entered the cell. In some aspects, such cells can comprise any of the cells described herein (e.g., stem cells or PBMCs, e.g., T cells or monocytes).

In some aspects, the present disclosure provides a composition comprising a plurality of cells, wherein the plurality of cells were produced by any of the methods provided herein. Also provided herein is a composition comprising a population of cells and a glial cell reprogramming factor under one or more parameters, which result in transient deformation of one or more cells of the population of cells and thereby creating perturbations in the cell membrane of the one or more cells, and wherein the perturbations in the cell membrane allows the glial cell reprogramming factor to enter the one or more cells.

Also provided are kits or articles of manufacture for use in delivering into a cell a glial cell reprogramming factor as described herein. In some aspects, the kits comprise the compositions described herein (e.g. a microfluidic channel or surface containing pores, cell suspensions, and/or glial cell reprogramming factor) in suitable packaging. Suitable packaging materials are known in the art, and include, for example, vials (such as sealed vials), vessels, ampules, bottles, jars, flexible packaging (e.g., sealed Mylar or plastic bags), and the like. These articles of manufacture can further be sterilized and/or sealed.

The present disclosure also provides kits comprising components of the methods described herein and can further comprise instruction(s) for performing said methods to deliver a glial cell reprogramming factor into a cell. The kits described herein can further include other materials, including other buffers, diluents, filters, needles, syringes, and package inserts with instructions for performing any methods described herein; e.g., instructions for delivering a glial cell reprogramming factor into a cell.

The following examples are offered by way of illustration and not by way of limitation.

### EXAMPLES

### Example 1: Selection of Cellular and Molecular Components for Transdifferentiation of Somatic Cells into Oligodendrocytes

To demonstrate that the methods provided herein can be used to produce oligodendrocytes, in some aspects, a specific blood cell population with the highest transdifferentiation competency to oligodendrocytes will be identified. Non-limiting examples of factors that will be considered in identifying the ideal cell population include: cell abundance, cell variability, reprogramming competency, and combinations thereof. Non-limiting examples of cell types for consideration include: bulk PBMCs (i.e., comprising both lymphocytes and myeloid cells, such as monocytes), purified population of T cells, purified population of monocytes, HSCs, or combinations thereof. In some aspects, using such cells, different reprogramming factors (e.g., those described herein) will be delivered to the cells using the squeezing methods provided herein. In some aspects, various combinations, delivery order, timing, and dosing of the different reprogramming factors will be tested. Then, using various assays (e.g., quantitative PCR, immunocytochemistry, transcriptomic analysis), the expression and activation of various genes associated with oligodendrocyte differentiation will be assessed.

### Example 2: Maturation and Functional Characterization of the Induced Oligodendrocytes

To assess the oligodendrocytes produced in Example 1, various approaches will be used to characterize both the maturation state and functional capability of the oligodendrocytes. For example, in some aspects, the maturation state of the oligodendrocytes will be assessed based on morphological structure and/or expression of markers associated with mature oligodendrocytes. In some aspects, functional aspects of the oligodendrocytes will be assessed in vitro, in vivo (e.g., rodent model of demyelination), or both.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific aspects have been illustrated and described, the above specification is not restrictive. It will be appreciated that various changes can be made without departing from the spirit and scope of the disclosure(s). Many variations will become apparent to those skilled in the art upon review of this specification.

1.-18. (canceled)

19. A method of inducing the reprogramming of a cell into a glial cell comprising:
passing a cell suspension, which comprises a population of cells, through a constriction under one or more parameters,
wherein passing the cell suspension through the constriction under the one or more parameters deforms one or more cells of the population of cells, and thereby, causing a perturbation in the cell membrane of the one or more cells, and
wherein the perturbation allows a glial cell reprogramming factor to enter the one or more cells, and thereby, induce the reprogramming of the one or more cells into a glial cell.

20. (canceled)

21. The method of claim 19, comprising contacting the population of cells with the glial cell reprogramming factor prior to, during or after passing of the cell suspension through the constriction.

22.-25. (canceled)

26. The method of claim 19, wherein the glial cell reprogramming factor comprises a nucleic acid, a polypeptide, a lipid, a carbohydrate, a small molecule, a metal-containing compound, an antibody, a transcription factor, a nanoparticle, a liposome, a fluorescently tagged molecule, or combinations thereof.

27.-36. (canceled)

37. The method of claim 19, wherein the one or more cells of the population of cells comprises stem cells, somatic cells, or both.

38.-45. (canceled)

46. The method of claim 19, wherein the one or more parameters are selected from a cell density; pressure; length, width, and/or depth of the constriction; diameter of the constriction; diameter of the cells; temperature;
entrance angle of the constriction; exit angle of the constriction; length, width, and/or width of an approach region; surface property of the constriction (e.g., roughness, chemical modification, hydrophilic, hydrophobic); operating flow speed; glial cell reprogramming factor concentration; viscosity, osmolarity, salt concentration, serum content, and/or pH of the cell suspension; time in the constriction; shear rate in the constriction; type of glial cell reprogramming factor, or combinations thereof.

47. The method of claim 46, wherein;
(i) the cell density is at least about 6×10⁷ cells/mL, at least about 7×10⁷ cells/mL, at least about 8×10⁷ cells/mL, at least about 9×10⁷ cells/mL, at least about 1×10⁸ cells/mL, at least about 1.1×10⁸ cells/mL, at least about 1.2×10⁸ cells/mL, at least about 1.3×10⁸ cells/mL, at least about 1.4×10⁸ cells/mL, at least about 1.5×10⁸ cells/mL, at least about 2.0×10⁸ cells/mL, at least about 3.0×10⁸ cells/mL, at least about 4.0×10⁸ cells/mL, at least about 5.0×10⁸ cells/mL, at least about 6.0×10⁸ cells/mL, at least about 7.0×10⁸ cells/mL, at least about 8.0×10⁸ cells/mL, at least about 9.0×10⁸ cells/mL, or at least about 1.0×10⁹ cells/mL or more;
(ii) the pressure is at least about 30 psi, at least about 35 psi, at least about 40 psi, at least about 45 psi, at least about 50 psi, at least about 55 psi, at least about 60 psi, at least about 65 psi, at least about 70 psi, at least about 75 psi, at least about 80 psi, at least about 85 psi, at least about 90 psi, at least about 95 psi, at least about 100 psi, at least about 110 psi, at least about 120 psi, at least about 130 psi, at least about 140 psi, or at least about 150 psi;
(iii) the diameter of the constriction is about 20%, about 30%, about 40%, about 50% about 60%, about 70%, about 80%, about 90%, or about 99% of the diameter of the one or more cells of the population of cells;
(iv) the length of the constriction is less than about 1 μm, less than about 5 μm, less than about 10 μm, less than about 20 μm, less than about 30 μm, less than about 40 μm, less than about 50 μm, less than about 60 μm, less than about 70 μm, less than about 80 μm, less than about 90 μm, or less than about 100 μm;
(v) the width of the constriction is less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm; and/or
(vi) the depth of the constriction is at least about 1 μm, at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm.

48.-62. (canceled)

63. The method of claim 19, wherein the population of cells are contacted with multiple glial cell reprogramming factors, such that at least two or more of the glial cell reprogramming factors are cable of entering the one or more cells after the perturbation of the cell membrane.

64. (canceled)

65. The method of claim 63, wherein the multiple glial cell reprogramming factors are delivered into the cells concurrently or sequentially.

66. (canceled)

67. The method of claim 19, comprising passing the cell suspension through a plurality of constrictions, wherein:
(i) each constriction of the plurality of constrictions are the same or one or more of the constrictions of the plurality of constrictions are different; and/or
(ii) each constriction of the plurality of constrictions is associated with the same glial cell reprogramming factor or one or more of the plurality of constrictions is associated with a different glial cell reprogramming factor.

68.-73. (Canceled)

74. The method of claim 67, wherein the plurality of constrictions comprise a first constriction associated with a first glial cell reprogramming factor and a second constriction associated with a second glial cell reprogramming factor, wherein the cell suspension passes through the first constriction such that the first glial cell reprogramming factor is delivered to one or more cells of the plurality of cells, and then the cell suspension passes through the second constriction such that the second glial cell reprogramming factor is delivered to the one or more cells of the plurality of cells.

75. (canceled)

76. The method of claim 19, further comprising contacting the plurality of cells with an additional compound, wherein:
(i) the additional compound comprises a nucleic acid, a polypeptide, a lipid, a carbohydrate, a small molecule, a metal-containing compound, an antibody, a transcription factor, a nanoparticle, a liposome, a fluorescently tagged molecule, or combinations thereof;
(ii) the contacting of the additional compound with the plurality of cells occurs concurrently with the glial cell reprogramming factor or occurs prior to or after the contacting of the plurality of cells with the glial cell reprogramming factor;
(iii) the additional compound is a signaling molecule that is capable of inducing the formation of a glial cell; and/or
(iv) the additional compound is a nucleic acid encoding an enzyme that confers resistance to an antibiotic.

77.-89. (canceled)

90. A composition comprising a population of cells and a glial cell reprogramming factor under one or more parameters resulting in deformation of one or more cells of the population of cells, wherein the one or more cells comprise a perturbation in the cell membrane sufficient to allow the glial cell reprogramming factor to enter the one or more cells.

91.-99. (canceled)

100. The composition of claim 90, wherein the glial cell reprogramming factor comprises a nucleic acid, a polypeptide, a lipid, a carbohydrate, a small molecule, a metal-containing compound, an antibody, a transcription factor, a nanoparticle, a liposome, a fluorescently tagged molecule, or combinations thereof.

101.-107. (canceled)

108. The composition of claim 90, wherein the one or more parameters are selected from a cell density; a pressure; a length, width, and/or depth of the constriction; a diameter of the constriction; a diameter of the cells; a temperature; an entrance angle of the constriction; an exit angle of the constriction; a length, width, and/or width of an approach region; a surface property of the constriction (e.g., roughness, chemical modification, hydrophilic, hydrophobic); an operating flow speed; a glial cell reprogramming factor concentration; a viscosity, osmolarity, salt concentration, serum content, and/or pH of the cell suspension; time in the constriction; shear rate in the constriction; type of glial cell reprogramming factor or combinations thereof.

109. The composition of claim 108, wherein:
(i) the cell density is at least about 6×10⁷ cells/mL, at least about 7×10⁷ cells/mL, at least about 8×10⁷ cells/mL, at least about 9×10⁷ cells/mL, at least about 1×10⁸ cells/mL, at least about 1.1×10⁸ cells/mL, at least about 1.2×10⁸ cells/mL, at least about 1.3×10⁸ cells/mL, at least about 1.4×10⁸ cells/mL, at least about 1.5×10⁸ cells/mL, at least about 2.0×10⁸ cells/mL, at least about 3.0×10⁸ cells/mL, at least about 4.0×10⁸ cells/mL, at least about 5.0×10⁸ cells/mL, at least about 6.0×10⁸ cells/mL, at least about 7.0×10⁸ cells/mL, at least about 8.0×10⁸ cells/mL, at least about 9.0×10⁸ cells/mL, or at least about 1.0×10⁹ cells/mL or more;
(ii) the pressure is at least about 30 psi, at least about 35 psi, at least about 40 psi, at least about 45 psi, at least about 50 psi, at least about 55 psi, at least about 60 psi, at least about 65 psi, at least about 70 psi, at least about 75 psi, at least about 80 psi, at least about 85 psi, at least about 90 psi, at least about 95 psi, at least about 100 psi, at least about 110 psi, at least about 120 psi, at least about 130 psi, at least about 140 psi, or at least about 150 psi;
(iii) the length of the constriction is less than about 1 μm, less than about 5 μm, less than about 10 μm, less than about 20 μm, less than about 30 μm, less than about 40 μm, less than about 50 μm, less than about 60 μm, less than about 70 μm, less than about 80 μm, less than about 90 μm, or less than about 100 μm;
(iv) the width of the constriction is less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm; and/or
(v) the depth of the constriction is at least about 1 μm, at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm.

110.-123. (canceled)

124. A cell comprising a perturbation in the cell membrane due to one or more parameters which deform the cell thereby causing the perturbation in the cell membrane of the cell such that a glial cell reprogramming factor can enter the cell.

125.-134. (canceled)

135. The cell of claim 124, wherein the glial cell reprogramming factor comprises a nucleic acid, a polypeptide, a lipid, a carbohydrate, a small molecule, a metal-containing compound, an antibody, a transcription factor, a nanoparticle, a liposome, a fluorescently tagged molecule, or combinations thereof.

136.-143. (canceled)

144. The cell of claim 124, wherein the one or more parameters are selected from a cell density; a pressure; a length, width, and/or depth of the constriction; a diameter of the constriction; a diameter of the cells; a temperature; an entrance angle of the constriction; an exit angle of the constriction; a length, width, and/or width of an approach region; a surface property of the constriction (e.g., roughness, chemical modification, hydrophilic, hydrophobic); an operating flow speed; a glial cell reprogramming factor concentration; a viscosity, osmolarity, salt concentration, serum content, and/or pH of the cell suspension; time in the constriction; shear rate in the constriction; type of glial cell reprogramming factor or combinations thereof.

145. The cell of claim 144, wherein:
(i) the cell density is at least about 6×10⁷ cells/mL, at least about 7×10⁷ cells/mL, at least about 8×10⁷ cells/mL, at least about 9×10⁷ cells/mL, at least about 1×10⁸ cells/mL, at least about 1.1×10⁸ cells/mL, at least about 1.2×10⁸ cells/mL, at least about 1.3×10⁸ cells/mL, at least about 1.4×10⁸ cells/mL, at least about 1.5×10⁸ cells/mL, at least about 2.0×10⁸ cells/mL, at least about 3.0×10⁸ cells/mL, at least about 4.0×10⁸ cells/mL, at least about 5.0×10⁸ cells/mL, at least about 6.0×10⁸ cells/mL, at least about 7.0×10⁸ cells/mL, at least about 8.0×10⁸ cells/mL, at least about 9.0×10⁸ cells/mL, or at least about 1.0×10⁹ cells/mL or more;
(ii) the pressure is at least about 30 psi, at least about 35 psi, at least about 40 psi, at least about 45 psi, at least about 50 psi, at least about 55 psi, at least about 60 psi, at least about 65 psi, at least about 70 psi, at least about 75 psi, at least about 80 psi, at least about 85 psi, at least about 90 psi, at least about 95 psi, at least about 100 psi, at least about 110 psi, at least about 120 psi, at least about 130 psi, at least about 140 psi, or at least about 150 psi;
(iii) the diameter of the constriction is about 20%, about 30%, about 40%, about 50%, about 60%, about 70%, about 80%, about 90%, or about 99% of the diameter of the one or more cells of the population of cells;
(iv) the length of the constriction is less than about 1 μm, less than about 5 μm, less than about 10 μm, less than about 20 μm, less than about 30 μm, less than about 40 μm, less than about 50 μm, less than about 60 μm, less than about 70 μm, less than about 80 μm, less than about 90 μm, or less than about 100 μm;
(v) the width of the constriction is less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm; and/or
(vi) the depth of the constriction is at least about 1 μm, at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm.

146.-173. (canceled)
